(19) United States
(12) Patent Application Publication
(10) Pub. No.: US 2024/0041551 A1
(43) Pub. Date: Feb. 8, 2024

(54) GUIDEWIRE HOLDER AND METHODS THEREOF

(71) Applicant: Biotex, Inc., Houston, TX (US)

(72) Inventors: Patrick Neville, Belleville, IL (US); Charles Houssiere, Houston, TX (US); Coleman Leach, Houston, TX (US); David Lawless, Houston, TX (US); Kim Najjar, Houston, TX (US); Ashok Gowda, Bellaire, TX (US)

(73) Assignee: Biotex, Inc., Houston, TX (US)

(21) Appl. No.: 18/364,980

(22) Filed: Aug. 3, 2023

Related U.S. Application Data
(60) Provisional application No. 63/370,342, filed on Aug. 3, 2022.

(51) Int. Cl. A61B 50/20; A61M 25/09; B08B 3/08; B08B 1/00

(52) U.S. Cl. CPC A61B 50/20; A61M 25/09; B08B 3/08; B08B 1/006; A61B 2050/002

(57) ABSTRACT

A holder for an elongated medical body suitable for insertion into a patient during a surgical procedure is provided. The holder includes a first outer member having a first inner surface, and a first outer surface, and a second outer member having a second inner surface and a second outer surface. The holder also includes a core extending between the first and second outer members, and at least one holding member placed between the inner surfaces of the first and second outer members. The holding member or holding members are compressible for holding the elongated medical body in the holder.

### FIELD OF THE INVENTION

The present invention relates to medical devices, and more specifically, to holders for medical devices.

### BACKGROUND

Medical devices for insertion into the body of the patient must remain sterile and undamaged prior to insertion. These medical devices, such as guidewires and catheters, are often stored on the patient or in the sterile field prior to use or during intermittent use. Further, wet sterile towels are conventionally used to hold these devices down and prevent them from falling or sliding off the patient. This covering prevents the user (e.g., medical professional) from seeing them, and knowing which devices are stored in which location. Also, during insertion and use, the users often require another person to hold the unused portion of the device to prevent it from falling to the floor.

When stored with other sterile devices in a procedure room, wires and devices are sometimes placed in buckets to hold them in a circular configuration and prevent them from being dropped on the floor. Multiple sizes and types of guidewires are sometimes used during procedures and can be stored together, and thus it can be difficult to tell which wire is which when preparing the devices for a procedure. Additionally, wires are typically coiled and then twisted inside of the coil, to prevent them from springing open and having parts or all of the wire land on the non-sterile floor, which necessitates replacement of the wire and adds cost to the procedure.

The present application addresses these and other challenges related to the holding or storing of medical devices intended for insertion into a patient.

### SUMMARY

In a first aspect, a holder for an elongated medical body suitable for insertion into a patient during a medical procedure is provided. The holder comprises a first outer member having a first perimeter, a first inner surface and a first outer surface, at least a portion of the first outer member lying in a first plane. The holder further comprises a second outer member having a second perimeter, a second inner surface and a second outer surface, at least a portion of the second outer member lying in a second plane. The holder also comprises a core extending between the first and second outer members at a location interior to the first and second perimeters, wherein the first and second outer members are maintained with the first and second inner surfaces in opposition about the core with the first and second planes oriented generally in parallel and spaced apart. The holder also comprises a compressible material disposed on at least a portion of one of the first and second inner surfaces, wherein the first and second inner surfaces are configured to engage and hold the elongated medical body when the elongated medical body is advanced therebetween towards the core and is in engagement with the compressible material.

In another aspect, the holder further comprises a writeable surface disposed on the outer surface of one of the first and second outer members.

In another aspect, the holder further comprises an adhesive disposed on one of the outer surfaces of the first and second outer members, wherein the adhesive is configured to attach to one of the first and second outer surfaces and to a surface not forming part of the holder.

In another aspect, the core further comprises a first connector attached to one of the first and second outer members, wherein the first connector is connected to the other of the first and second outer members.

In another aspect, the first and second inner surfaces are configured to form a funnel cross sectional shape.

In a second aspect, a holder for an elongated medical body suitable for insertion into a patient during a medical procedure is provided. The holder comprises a first outer member having a first inner surface and a first outer surface, and a second outer member having a second inner surface and a second outer surface. The holder also comprises a core structure extending between the first and second outer members, and an adhesive member configured to adhere the holder to a first surface. The holder also comprises two holding members respectively placed between the inner surfaces of the first and second outer members, and wherein the holding members are compressible and configured to hold therebetween the elongated medical body. The adhesive member is configured to attach to one of the first and second outer surfaces. The elongated medical body may be, for example, a guidewire or a catheter or a surgical tool or an electrode for stimulating tissue or the like.

In another aspect, the core structure comprises a first connection member coupled to the first outer member and a second connection member coupled to the second outer member.

In another aspect, the holder further comprises a writable surface on the first and second outer surfaces.

In another aspect, each of the first and second outer members have an outer perimeter and a center and a surface that tapers from the outer perimeter to a point between the outer perimeter and the center defining a funnel configured to capture the elongated medical body.

In another aspect, the attachment of the first and second outer members defines a space suitable for holding the elongated medical body between the two holding members.

In another aspect, the two holding members further comprise a foam material.

In another aspect, the two holding members further comprise a silicone material. In a further aspect, at least one of the holding members further comprise a plurality of protrusions, wherein the protrusions are configured to capture portions of the elongated medical body.

In a further aspect, at least some of the protrusions are of differing sizes.

In another aspect, at least one of the first and second outer members includes a recess on its outer surface relative to its outer edge, wherein the recess comprises a recess surface configured to receive labeling or the adhesive member.

In another aspect, the first connection member comprises two prongs and the second connection member comprises two openings, wherein the two prongs are configured to securely fit into and attach to the two openings.

In another aspect, the first connection member comprises a protruding member and the second connection member comprises a notch configured to securely receive the protruding member, wherein the first outer member and the second outer member are securely attached to one another having an axis of rotation, and in a locked position upon insertion of the protruding member into the notch and subsequent turning of either the first or the second outer member in a first direction about the axis of rotation. In a further aspect, the protruding member comprises a compressible member configured to assist in releasing the first outer member and the second outer member from the locked position upon turning either the first or the second outer member in a second direction about the axis of rotation. In a further aspect, the compressible member is a spring.

In another aspect, the writable surface comprises a label adhered to the outer surface of the respective outer member.

In another aspect, the adhesive member maintains its adhesive qualities upon multiple attachments and detachments from the first surface.

In another aspect, the adhesive member is configured to removably attach to the first surface, and wherein the first surface is a drape, a garment, a table, or surgical tray.

In another aspect, the holder further comprises a secondary attachment mechanism attached to either the first outer member or the second outer member, wherein the secondary attachment mechanism is configured to attach to a drape or a garment.

In a third aspect, a method for operating a holder for an elongated medical body is provided. The method comprises a step of providing a holder, wherein the holder comprises: a first outer member having a first inner surface and a first outer surface; a second outer member having a second inner surface and a second outer surface; a core structure extending between the first and second outer members; an adhesive member; and two holding members respectively placed between the inner surfaces of the first and second outer members. Each of the first and second outer members have an outer perimeter and a surface that tapers from the outer perimeter to a point on the respective inner surface defining a funnel configured to capture the elongated medical body. The method further comprises the steps of feeding a portion of the elongated medical body, via the funnel, between the two holding members, and holding the elongated medical body between the two holding members via friction.

In another aspect, at least one of the first and second outer members includes a recess on its outer surface relative to its outer edge, wherein the recess is configured to receive a label having a writable surface or the adhesive member.

In another aspect, the method further comprises the step of attaching the adhesive member to a first surface. In a further aspect, the adhesive member is configured to removably attach to the first surface, and wherein the first surface is a drape, a garment, a table, or surgical tray.

In another aspect, the elongated medical body is a guidewire. In a further aspect, the method further comprises the step of: pulling the guidewire to cause a portion of the guidewire held between the two holding members to be released, wherein the pulling of the guidewire causes the portion of the guidewire to be cleaned by the two holding members as it is released. In a further aspect, the method further comprises the step of: applying a cleaning liquid on the holding members.

In a fourth aspect, a kit is provided. The kit can include: a holder for an elongated medical body as described above, a writing utensil configured to write on a writable surface of the holder, a removeable covering for the adhesive member of the holder, and an instrument pad for wiping an elongated medical body.

In another aspect, the instrument pad is an antiseptic instrument pad.

In another aspect, the kit further comprises at least one elongated medical body, wherein the at least one elongated medical body is a guidewire or a catheter.

In another aspect, the kit further comprises a pouch card configured for selective attachment to the holder, wherein the pouch card is removably fastened to an attachment mechanism.

### BRIEF DESCRIPTION OF THE DRAWINGS

FIGS. 1A-1H disclose various views of a holder for a medical cylindrical body in accordance with one or more embodiments; FIG. 1A—front view of the first and second outer members; FIG. 1B—front perspective view of the first outer member; FIG. 1C side views of the first outer member; FIG. 1D—cross-sectional view of the first outer member; FIG. 1E—front and front perspective views of the holder; FIG. 1F—side views of the holder; FIG. 1G—exploded view and exploded perspective view of the holder; FIG. 1H—front perspective view of the holder holding a guidewire.

FIGS. 2A-2H disclose various views of a holder for a medical cylindrical body in accordance with one or more other embodiments; FIG. 2A—front view of the first and second outer members; FIG. 2B—front perspective view of the first outer member; FIG. 2C—side views of the first outer member; FIG. 2D—cross-sectional view of the first outer member; FIG. 2E—front and side views of the holder; FIG. 2F—side view of the holder (left image) and front perspective views of the holder (center image) and the first outer member with the holding member (right image); FIG. 2G—exploded view and exploded perspective view of the holder; FIG. 2H—top perspective and back perspective view of holder attached to a medical drape.

FIGS. 3A-3J disclose various views of a holder for a medical cylindrical body in accordance with one or more other embodiments; FIG. 3A—front view of the first and second outer members; FIG. 3B—front perspective views of the first outer member and second outer member; FIG. 3C—vertical side views of the first outer member and second outer member; FIG. 3D—horizontal side views of the first outer member and second outer member; FIG. 3E—front and front perspective views of the holder; FIG. 3F—side views of the holder in a closed position; FIG. 3G—side views of the holder in an open position; FIG. 3H—bottom perspective view of the locking mechanisms of the first and second outer members; FIG. 3I—exploded view and exploded perspective view of the holder; FIG. 3J—top perspective and front perspective views of the holder attached to a medical drape, holding a guidewire, and comprising a label.

FIGS. 4A-4C show exemplary embodiments of the attachment of the holder to the medical drape or other supporting surface via an adhesive on a surface of the holder.

FIGS. 5A-5C display exemplary embodiments of the holder in which the top outer comprises a toggle button on its outer surface to selectively lock and unlock a clamping feature in accordance with one or more embodiments.

FIGS. 5D-5E display a securing mechanism having a “pop” button integrated into the top outer member in accordance with one or more embodiments.

FIGS. 5F-5G display a securing mechanism having a squeeze locking mechanism integrated into the top outer member in accordance with one or more embodiments.

FIGS. 5H-5I display a securing mechanism that includes a latch for latching an end of the top outer member to an end of the bottom outer member in accordance with one or more embodiments.

FIGS. 5J-5K display a securing mechanism that includes a plurality of locking tabs 507 on the edges of the bottom outer member in accordance with one or more embodiments.

FIGS. 5L-5M display a securing mechanism in which the top outer member is rotated to lock or unlock the holder in accordance with one or more embodiments.

FIGS. 5N-5P display a rotating securing mechanism in which each of the first and second outer members include gear-like protrusions at regular intervals along their respective inner surfaces in accordance with one or more embodiments.

FIGS. 5Q-5R display a rotating securing mechanism in which the top and bottom outer members are substantially square-shaped.

FIGS. 6A-6B display a holder of the present application that is attached to a pouch card with a secondary attachment mechanism in accordance with one or more embodiments.

FIGS. 6C-6D display a holder of the present application that includes a secondary attachment mechanism in the form of a ratchet clip in accordance with one or more embodiments.

FIGS. 6E-6F display a holder of the present application that includes one or more integrated teeth as a secondary attachment mechanism in accordance with one or more embodiments.

FIGS. 6G-6H display a holder of the present application that includes a hole molded into one of the outer members through which a secondary attachment mechanism (e.g., a clip) can be attached, in accordance with one or more embodiments.

FIGS. 6I-6J display a holder of the present application that includes a spring-action clip attached to one of the outer members, which acts as a seconding attachment mechanism in accordance with one or more embodiments.

### DETAILED DESCRIPTION

The present application describes holders for a medical cylindrical body. The present application further describes methods for operating a holder for a medical cylindrical body and kits comprising a holder for a medical cylindrical body, such as a guidewire or a catheter. The holder for a medical cylindrical body of the present application can include first and second outer members, which can be made of plastic or metal, for example, and each have a connection member that securely attaches to one another. Each of the outer members includes an inner surface and an outer surface. In one or more embodiments, the first and second outer members can be circular, square, oval, or polygon shaped, for example.

The first and second outer members can each include a perimeter, and at least a portion of the first outer member lies in a first plane while at least a portion of the second outer member lies in a second plane. In one or more embodiments, the outer members are structures that have a surface area and thickness, in which the thickness is less than the surface area. In one or more embodiments, the outer members can vary in height, width, diameter, and/or thickness (and thus are not uniform) along the length of the outer member. The two outer members can have matching dimensions or, in certain embodiments, can have varying dimensions.

A core structure can extend between the first and second outer members at a location interior to the perimeters of the first and second outer members. In one or more embodiments, the core can be integrated into the first and/or second outer members. In at least one embodiment, the core can be couplable to the first and second outer members. In at least one embodiment, the core can comprise a first connection member coupled to the first outer member and a second connection member coupled to the second outer member, and the respective connection members can be configured to securely couple to one another. The first and second outer members can be oriented such that their respective inner surfaces are in opposition to one another about the core, with the first and second planes oriented generally in parallel and spaced apart.

In one or more embodiments, the first outer member can include a surface configured to be written on for inclusion of information thereon, e.g., information relevant to the involved medical procedure and/or the medical device(s), the cylindrical body held by the holder, or the patient. In at least one alternative embodiment, the first outer member can include a label for marking information thereon. The second outer member can include an adhesive on its outer surface that is configured to removably attach to a surgical drape of a patient. The holder can further include at least one holding member that can be made of a softer material (e.g., compressible material) than the outer members, such as a plastic foam, open or closed cell plastic material, or other soft polymers, where the at least one holding member is placed between the inner surfaces of the first and second outer members. For example, a holding member can be disposed on least a portion of one of the inner surfaces of the first and second outer members, or a portion of both of them, or on all of the inner surfaces.

In one or more embodiments, the attachment of the first and second outer members to each other defines a space for holding the elongated medical body between the two holding members. In at least one embodiment, upon attachment of the first and second outer members, the two holding members are in contact with one another. In one or more embodiments, the outer members both taper from an edge of the outer surface to a point on the inner surface (e.g., the edge of the inner surface) to define an annular funnel, which allows the holder to more easily receive and capture the elongated medical body therein. At least a portion of the medical cylindrical body can then be fed via the funnel to the holding member(s), e.g., successively capturing successive loops of the medical cylindrical body held therein, and the medical cylindrical body is then held between the holding members via friction (or between a holding member and an inner surface of one of the outer members via friction). The holding members are compressible such that as the medical cylindrical body is pushed into the device, the medical cylindrical body slides between the holding members and is held by the compression of the holding members against the more rigid outer members. During use of the cylindrical body during a procedure, as a portion of the medical cylindrical body is pulled from the holder, the pulling of the medical cylindrical body can cause a portion of the medical cylindrical body to be cleaned by the two holding members as it is released. In one or more embodiments, the holding members can be wetted with a sterile or sterilizing fluid such that as the portion of the medical cylindrical body is pulled from the holder, the medical cylindrical body is moistened (e.g., for lubricity before insertion into the patient) or cleaned by the fluid.

In at least one embodiment, the holder can include outer members without the two holding members, and in such an embodiment, the tapering of the outer members captures medical cylindrical body, which is then held between the outer members.

The holder of the present application allows a medical professional (e.g., surgeon) to perform a medical procedure requiring insertion of the medical cylindrical body into the patient without needing another assistant to hold the external length of the medical cylindrical body while they are inserting it, thereby preventing accidental dropping or contamination of the medical cylindrical body.

As further shown and described herein, the present apparatuses and methods provide an efficient solution for holding elongated medical bodies (e.g., medical cylindrical bodies) of varying sizes and diameters (e.g., guidewires, catheters, and thin tools insertable into a patient that are used in minimally invasive medical procedures). Additionally, in one or more embodiments, the holder of the present application can hold multiple medical cylindrical bodies at one time.

Additionally, while the elongated medical bodies held by the holder of the present application are generally described herein as generally “cylindrical” in shape, the medical bodies can also be of other shapes, including elongated shapes that are non-uniform in cross section and/or dimension. As used herein, the terms “medical cylindrical body,” “cylindrical body” and “elongated medical body” are used interchangeably, but it is noted that “medical cylindrical body” or “cylindrical body” is one preferred embodiment of an elongated medical body.

The present holder and methods for operating a holder for a medical cylindrical body are now described more fully with reference to the accompanying drawings, in which one or more illustrated embodiments and/or arrangements of the methods are shown. The apparatuses and methods are not limited in any way to the illustrated embodiments and/or arrangements as the illustrated embodiments and/or arrangements described below are merely exemplary of the methods, which can be embodied in various forms, as appreciated by one skilled in the art. Therefore, it is to be understood that any structural and functional details disclosed herein are not to be interpreted as limiting the apparatuses and methods, but rather, are provided as a representative embodiment and/or arrangement for teaching one skilled in the art one or more ways to implement the apparatuses and methods. Furthermore, the terms and phrases used herein are not intended to be limiting, but rather are to provide an understandable description of the apparatuses and methods. Additionally, as used in the present application, the term “approximately” when used in conjunction with a numerical value refers to any number within about 5, 3 or 1% of the referenced numerical value, including the referenced numerical value. As used in the present application, the term “generally” or “substantially” or “approximately” in reference to the orientation of two objects (e.g., generally parallel, generally in parallel, or substantially parallel to one another) refers to any value within about 0-20 degrees of the specified term (e.g., within about 0-20 degrees of parallel).

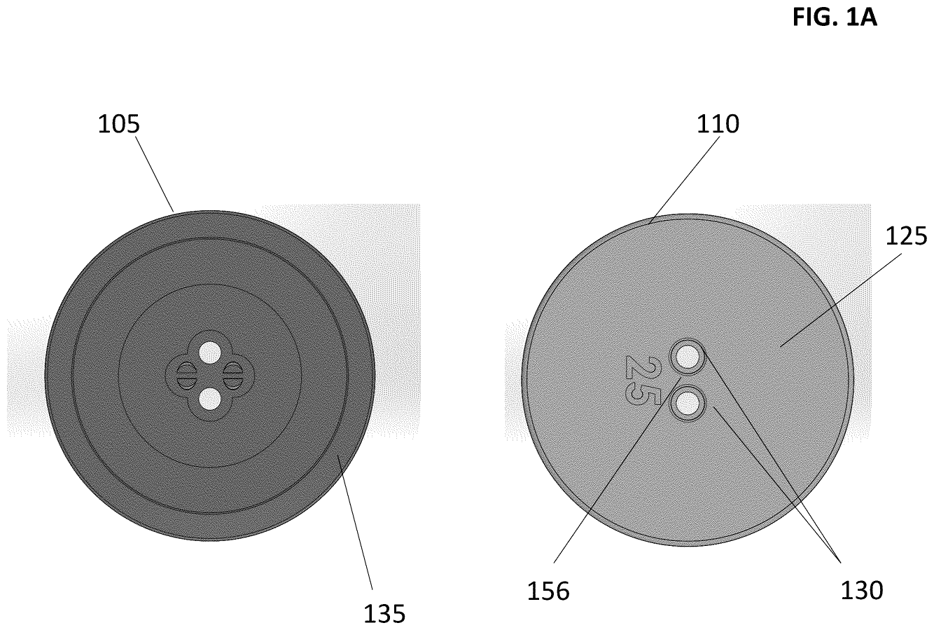

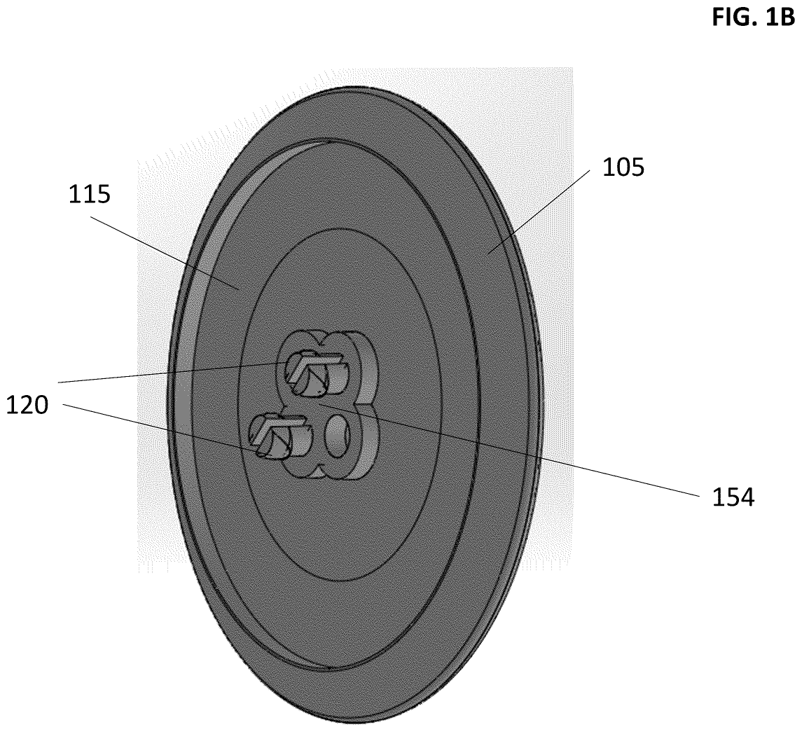

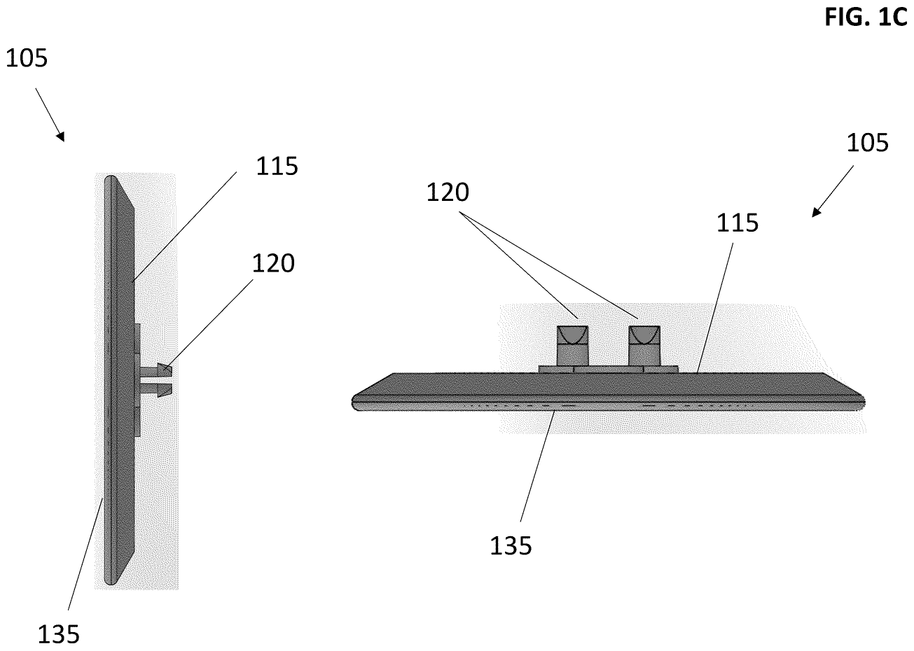

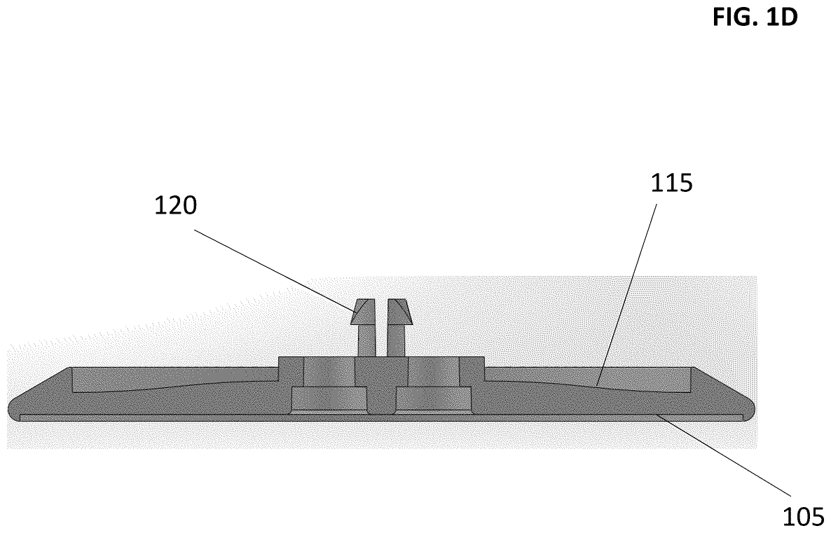

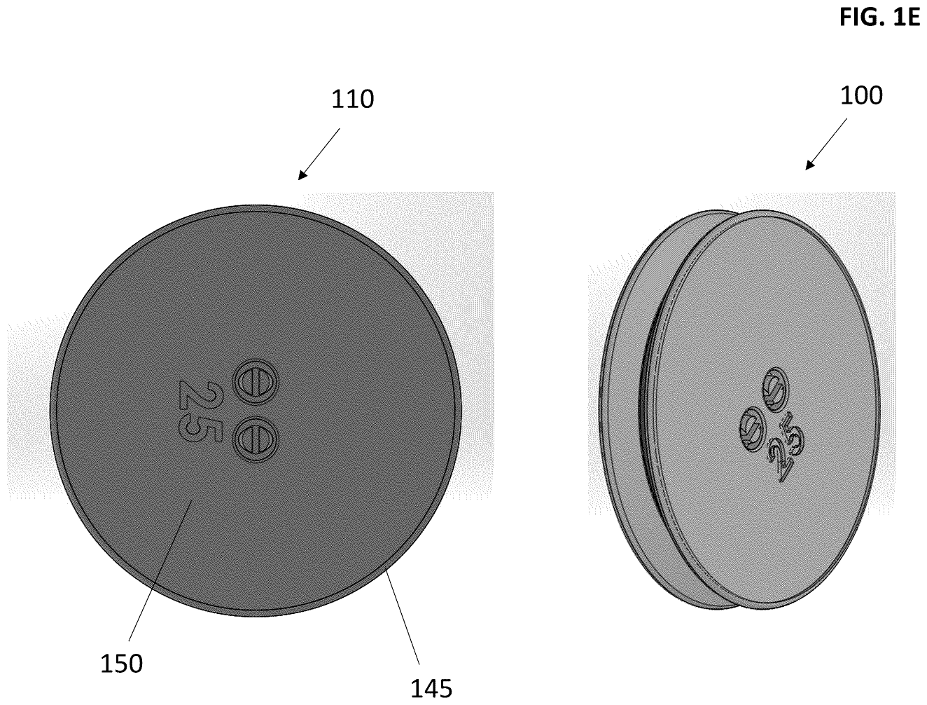

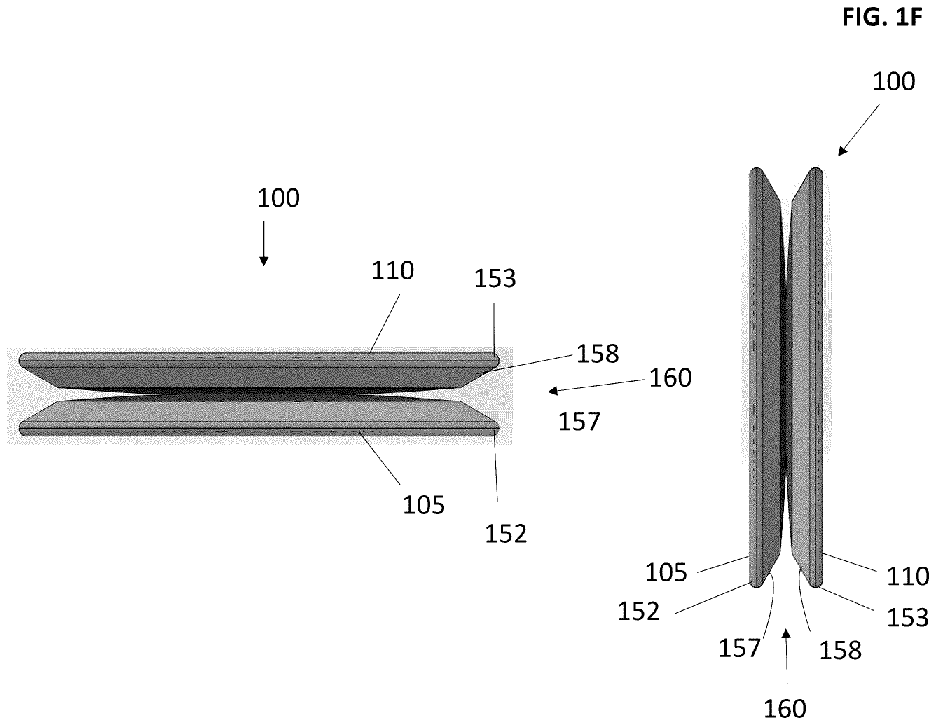

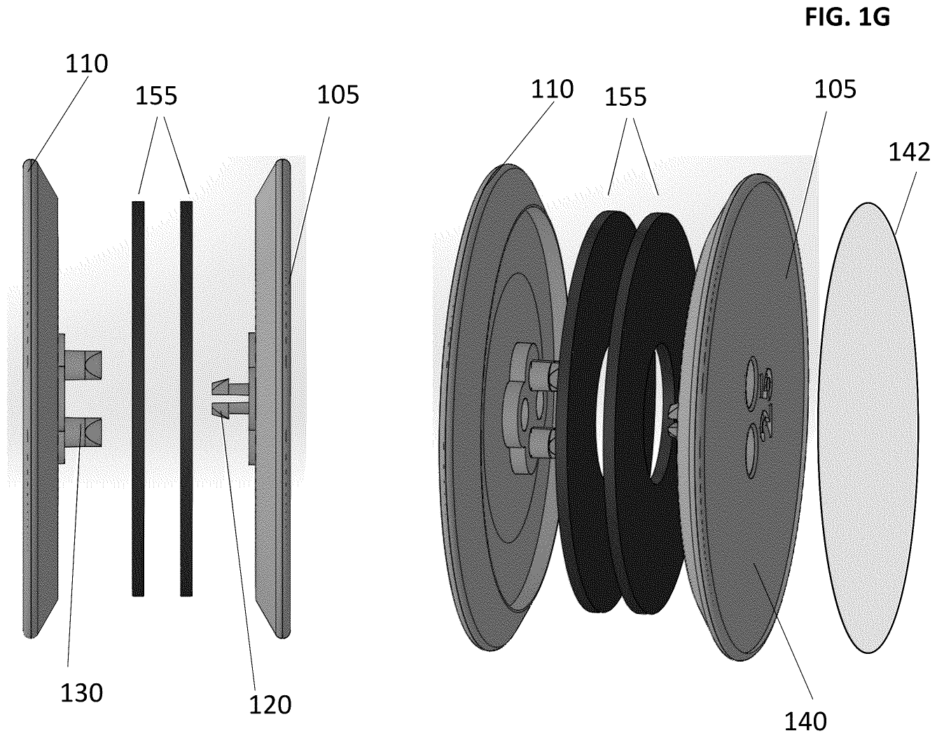

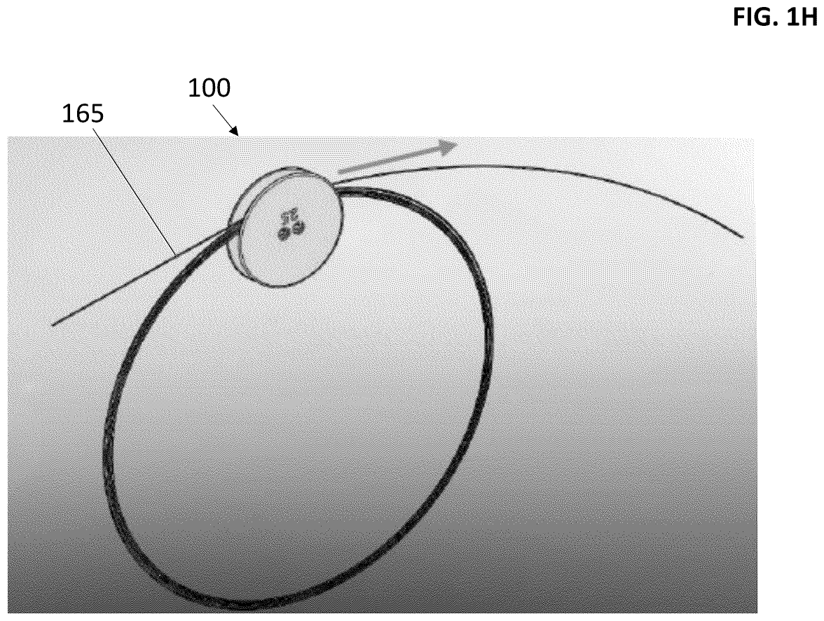

FIGS. 1A-1H show various views of a holder 100 for a medical cylindrical body in accordance with one or more embodiments. With reference now to FIGS. 1A-1D, the holder 100 includes a first outer member 105 and a second outer member 110. The first and second outer members 105 and 110 can be made of a plastic material, metal, or a similarly durable material. The first outer member 105 includes an inner surface 115 having a first connection member 120. As shown in FIGS. 1A-1D, in one or more embodiments, the first connection member 120 can be two prongs or prong-like appendages. The second outer member 110 also includes an inner surface 125 which comprises a second connection member 130. In one or more embodiments, as exemplified in FIG. 1A, the second connection member 130 can be two openings, and the two prongs (first connection member 120) are sized and shaped to securely fit into and attach to the respective openings. For example, in the embodiment of FIGS. 1A-1H, upon insertion of the prongs 120 through the respective openings 130, the protruding edges of the prongs can latch onto the edges of the openings 130 to provide a secure attachment between the prongs and the openings. In other embodiments, prongs 120 may be provided on one outer member, with corresponding openings on the other outer member. It should be understood that, in at least one embodiment, the first and second connection members 120 and 130 can attach to each other via other mechanisms such screws, adhesives, or press fit attachments, for example.

With continued reference to FIGS. 1A-1H, the first outer member 105 also includes an outer surface 135 that optionally comprises a recess 140. In one or more embodiments, the outer surface 135 or if present recess 140 can be configured to receive an adhesive member 142. In one or more embodiments, the adhesive member 142 is a double-sided adhesive member, where one side attaches to the outer surface 135 or recess 140 and the other to the surface of an object. The adhesive member 142 allows the holder to be selectively detachable and re-attachable to the object. In one or more embodiments, the object is a medical drape, and the adhesive member 142 is specifically configured to be detachable and re-attachable to the medical drape.

In many medical procedures in which one or more medical cylindrical bodies (e.g., guidewire, catheter) are inserted into a patient's body, such as a heart or any vascular catheterization procedure, portions of the patient's body are covered by a medical (surgical) drape. Reusable surgical drapes are typically made of a woven material that can be cleaned and sterilized before reuse. Disposable (single use) drapes are typically made of a non-woven material (e.g., paper-based with a liquid resistance film). As such, in one or more embodiments, the adhesive member 142 on the outer surface 135 or recess 140 is specifically designed to be selectively detachable and re-attachable to the material of a reusable surgical drape and/or a disposable surgical drape. While the adhesive member 142 can be selectively detached and re-attached to the drape, the attachment strength of the adhesive member 142 is of sufficient strength to remain attached to the drape and the holder 100 during normal storing, pushing, and pulling of the medical cylindrical body in the holder 100 (e.g., guidewire) during a medical procedure. The adhesive member 142 allows the holder to be attached to the surgical drape in any direction or orientation. The outer surface 135 or the recess 140 is flat or substantially flat such that attaching it to the medical drape via the adhesive member 142 keeps the holder 100 attached as closely as possible and at a low profile relative to the medical drape and the holder 100 is thus less likely to be dislodged or moved accidentally during a medical procedure. While as described herein, the holder 100 is typically described as adhering to the medical drape of the patient, it should be understood that the holder can also be configured to attach to the medical drape or garment of a medical professional or technician, or other objects, such as a table or a surgical tray. Additionally, in one or more embodiments, the holder can be selectively and removably attached to one or more different surfaces (e.g., drape, table, etc.) via the adhesive member during use of the holder (e.g., during a medical procedure).

In one or more embodiments, the adhesive member 142 is any adhesive that can be sterilized and can adhere strongly enough to the medical drape such that it can be removed and reapplied to the drape multiple times. For example, the adhesive member 142 can be a sterilizable, double-sided, tackified acrylic adhesive on a transparent polyethylene carrier. In at least one embodiment, the adhesive member 142 is double coated medical polyethylene film tape, such as 3M Product Number 1509. The one side of the adhesive member 142 adheres strongly to the outer surface 135 or recess 140 of the holder 100, and the other side of the adhesive member 142 is removably adherable to the medical drape.

Like the first outer member 105, in one or more embodiments, the second outer member 110 includes an outer surface 145 that optionally comprises a recess 150 (see FIG. 1E). In one or more embodiments, the outer surface 145 or the recess 150 can be configured to be written on. Specifically, the outer surface 145 or the recess 150 can comprise a material that allows for writing via a marker, pen, or pencil for example. A physician or medical professional can write on the recess to denote various aspects of, e.g., the medical cylindrical body to be held by the holder 100 or the patient that is being operated on, such as the size of the medical cylindrical body (e.g., 0.035-inch guidewire), the patient's name, or the procedure to be performed on the patient, or which medical practitioner is responsible for the item held in the holder. As such, the outer surface 145 or the recess 150 can be a flat or substantially flat surface to allow for legible writing.

In at least one alternative embodiment, instead of the outer surface 145 or the recess 150 being configured to be written on, the outer surface 145 or the recess 150 can be configured to receive a label that is of a material that allows for writing via a marker, pen, or pencil for example.

While as described above, the adhesive member is described as being configured for attachment to the outer surface 135 and the outer surface 145 is described as being configured to be written on or to receive a label for being written on (i.e., a “writable surface”), it should be understood that in certain embodiments, the adhesive can be configured for attachment to the outer surface 145 and the writable surface can be outer surface 135.

Accordingly, as described throughout the specification, in certain embodiments, the adhesive member is configured to be attached to the outer surface of the first outer member or the outer surface of the second outer member, and the writable surface is the outer surface of the first outer member or the second outer member (or a corresponding label).

With reference to FIG. 1G, in one or more embodiments, the holder 100 further includes two holding members 155 placed between the inner surface 115 of the first outer member 105 and the inner surface 125 of the second outer member 130. Each of the holding members 155 includes a throughhole for accommodating the first connection member 120 and/or the second connection member 130 upon attachment of the first and second outer members. In one or more embodiments, the holding members 155 can be adhered to the respective inner surfaces of the outer members (e.g., via an adhesive) and provide friction against the medical cylindrical body. In at least one embodiment, the holding members 155 can rotate freely within the holder upon attachment of the connection members 120 and 130. Upon attachment of the first and second outer members via their respective connection members 120 and 130, a space for holding the medical cylindrical body can be defined between the two holding members 155. In at least one embodiment, there is no space between holding members 155 when the medical cylindrical body is not within the holder 100, but because the holding members are compressible, once the medical cylindrical body is fed into the holder 100, the holding members 155 create the space for the medical cylindrical body between them. The holding members 155 are configured to hold the medical cylindrical body between them via tension when the first and second outer members are attached. The holding members 155 can be made of foam (e.g., plastic foam, open or closed cell plastic material, or other soft polymers), silicone, rubber or a similar deformable or flexible material that can securely hold the medical cylindrical body but without damaging the medical cylindrical body.

In one or more embodiments, the holding members 155 can be wetted with an antiseptic or antibacterial liquid such that upon pulling the medical cylindrical body (e.g., guidewire) through the holder 100, the medical cylindrical body glides along the surfaces of the holding members 155 thereby sterilizing or further cleaning the medical cylindrical body prior to insertion into the body. In one or more embodiments, the holding members 155 may also be wetted by the user with a sterile saline or water for cleaning or wetting of the medical cylindrical body.

The first and second outer members 105 and 110 and the holding members 155 can be sized and shaped to fit specific types of medical cylindrical body, and in particular, medical cylindrical bodies of varying sizes. For example, in the case of guidewires, the first and second outer members 105 and 110 and the holding members 155 can be sized and shaped to securely hold an 035 guidewire (guidewire of a diameter of 0.035 inches) or alternatively an 014 guidewire (guidewire of a diameter of 0.014 inches), for instance.

As exemplified in FIGS. 1F, both the first and second outer members 105 and 110 taper from an edge of the respective outer surfaces to the edge of the respective inner surfaces defining an annular funnel 160 configured to capture the medical cylindrical body. More specifically, each of the first and second outer members 105 and 110 have respective outer perimeters 152 and 153, and respective centers 154 and 156 (see FIGS. 1A and 1B) and respective surfaces 157 and 158 that taper from the outer perimeter 152, 153 to a point between the outer perimeter 152, 153 and the center 154, 156 defining the annular funnel 160 configured to capture the medical cylindrical body. The funnel shape of the outer members also helps with the ability of the holder 100 to clean the medical cylindrical body. Specifically, the holding members of the holder 100 grip the cylindrical body 165 (e.g., guidewire) in a static state, but when force is applied by the operator, such as by pulling on the wire, the wire is able to pass through (see FIG. 1H). As the wire passes through, the funnel shape of the holder 100 will naturally filter out large debris and small debris will be cleaned off as the wire passes through the holding members. Fluids can be absorbed into the holding members, thereby fully cleaning the medical cylindrical body (e.g., guidewire).

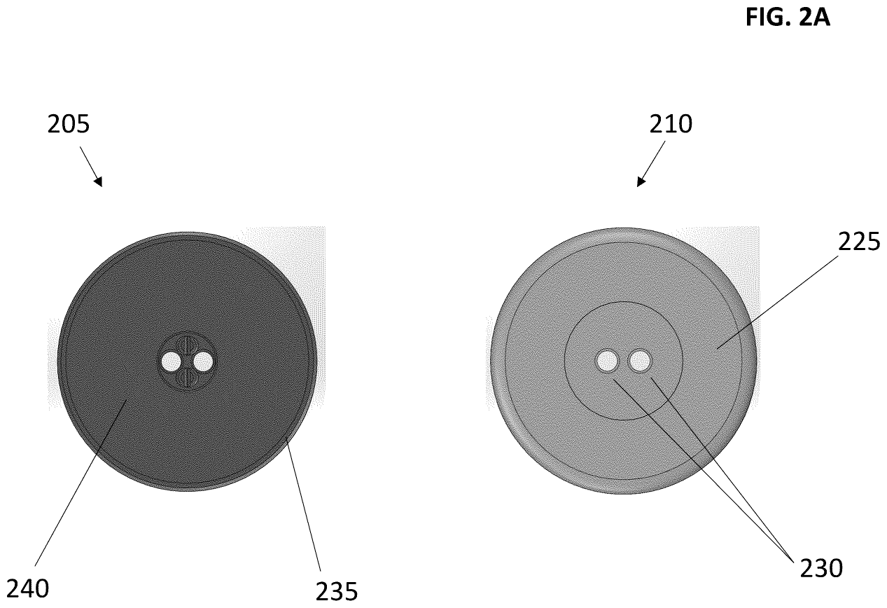

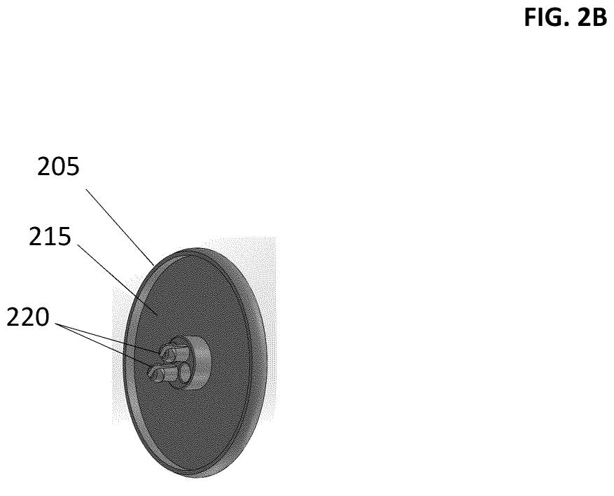

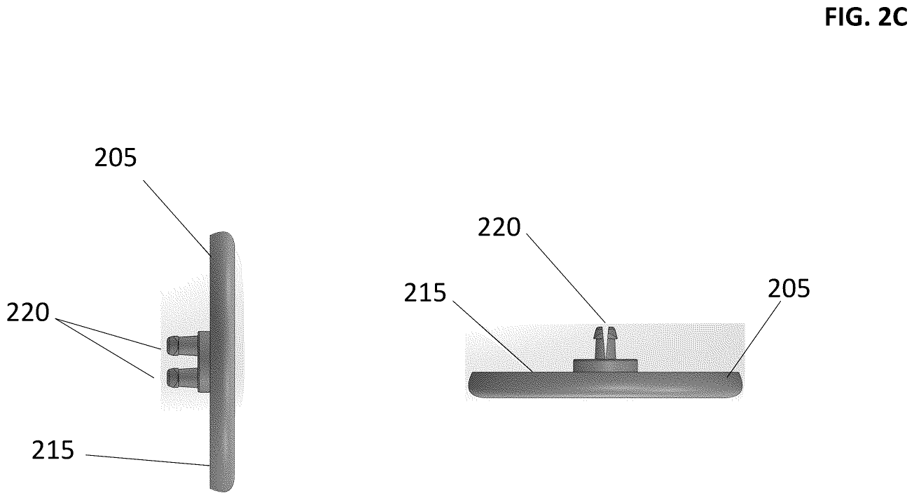

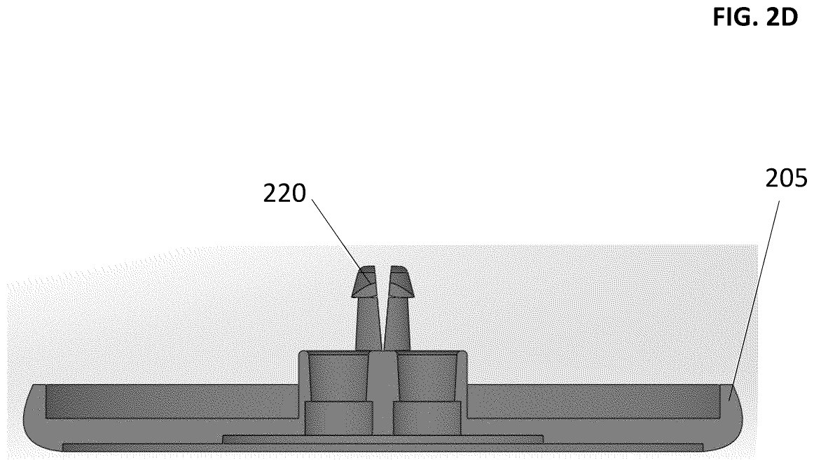

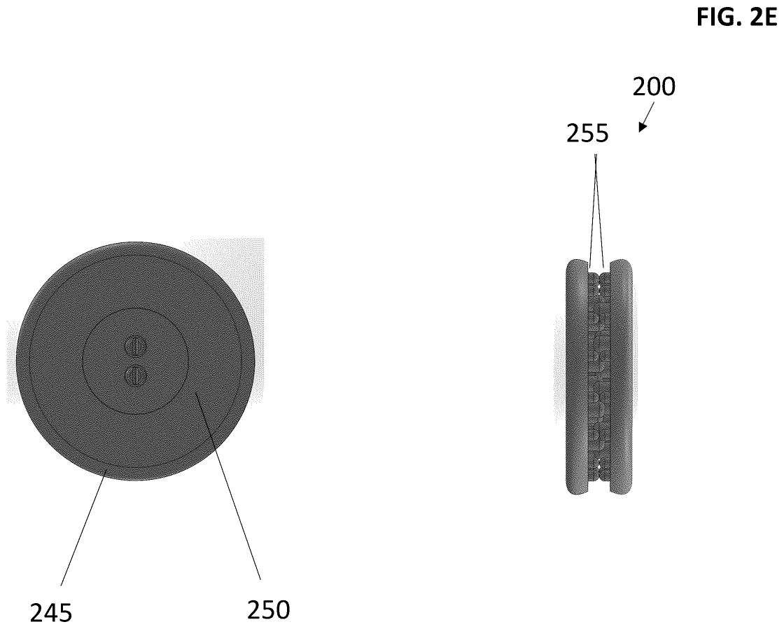

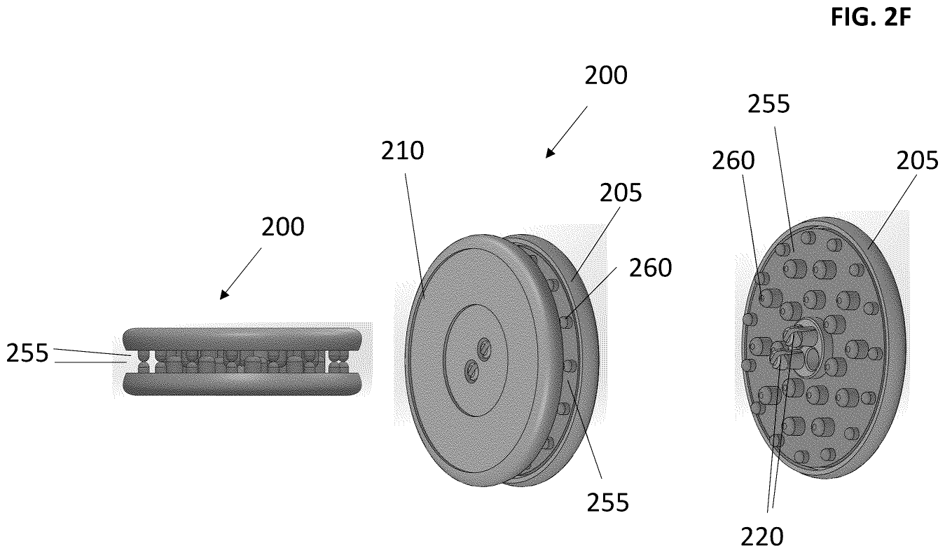

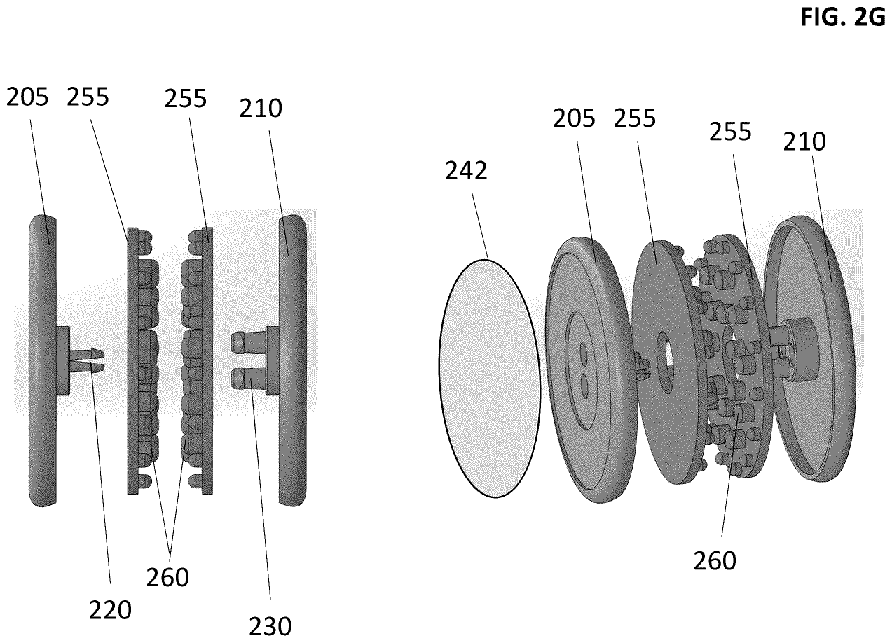

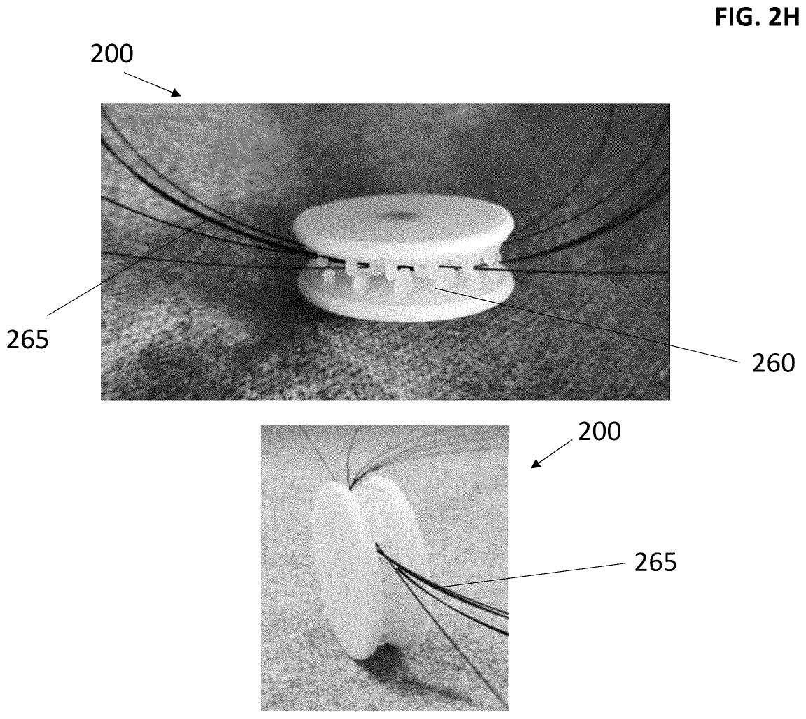

FIGS. 2A-2H disclose another embodiment of the holder for a medical cylindrical body of the present application, having similar components as the embodiment exemplified in FIGS. 1A-1H, with similar attributes unless otherwise noted. With reference now to FIGS. 2A-2H, the holder 200 includes a first outer member 205 and a second outer member 210, which can be made of a plastic material, metal, or a similarly durable material. The first outer member 205 includes an inner surface 215 having a first connection member 220. As shown in FIGS. 2B-2D, in one or more embodiments, the first connection member 220 can be two prongs or prong-like appendages. The second outer member 210 also includes an inner surface 225 which includes a second connection member 230. In one or more embodiments, as exemplified in FIG. 2A, the second connection member 230 can be two openings, and the two prongs (first connection member 220) are sized and shaped to securely fit into and attach to the respective openings. For example, in the embodiment of FIGS. 2A-2H, upon insertion of the prongs 220 through the respective openings 230, the protruding edges of the prongs can latch onto the edges of the openings 230 to provide a secure attachment between the prongs and the openings. In another embodiment, one outer member can have a prong and an opening with the other member having a corresponding opening and prong for securing the two outer members together. It should be understood that, in at least one embodiment, the first and second connection members 220 and 230 can attach to each other via other mechanisms such screws, adhesives, or press fit attachments, for example.

With continued reference to FIGS. 2A-2H, the first outer member 205 also includes an outer surface 235 that optionally comprises a recess 240. The outer surface 235 or the recess 240 can be configured to receive an adhesive member 242. In one or more embodiments, the adhesive member 242 is a double-sided adhesive member, where one side attaches to the outer surface 235 or the recess 240 and the other to the surface of an object (e.g., medical drape). In one or more embodiments, the adhesive member 242 is selectively detachable and re-attachable to the medical drape. The outer surface 235 or the recess 240 is flat or substantially flat such that attaching it to the medical drape via the adhesive member 242 keeps the holder 200 attached as closely as possible and at a low profile relative to the medical drape.

The second outer member 210 includes an outer surface 245 that optionally comprises a recess 250, either of which can be configured to be written on. Specifically, the outer surface 245 or recess 250 can comprise a material that allows for writing via a marker, pen, or pencil for example, such that a medical professional can write on the outer surface or recess to denote various aspects related to the medical cylindrical body, the procedure, or the patient. The outer surface 245 or the recess 250 can be a flat or substantially flat surface to allow for legible writing. In at least one alternative embodiment, instead of the outer surface 245 or recess 250 being configured to be written on, the outer surface 245 or recess 250 can be configured to receive a label that is of a material that allows for writing.

With reference to FIGS. 2E-2G, in one or more embodiments, the holder 200 further includes two holding members 255 placed between the inner surface 215 of the first outer member 205 and the inner surface 225 of the second outer member 230. Each of the holding members 255 includes a throughhole for accommodating the first connection member 220 and/or the second connection member 230 upon attachment of the first and second outer members.

Upon attachment of the first and second outer members via their respective connection members 220 and 230, a space for holding the medical cylindrical body can be defined between the two holding members 255 or can be defined between the holding members 255 upon insertion of the medical cylindrical body. The holding members 255 are configured to hold the medical cylindrical body between them via tension when the first and second outer members are attached. The holding members 255 can be made of foam (e.g., plastic foam or open or closed cell plastic material), silicone, rubber or a similar deformable or flexible material that can securely hold the medical cylindrical body but without damaging the medical cylindrical body.

In one or more preferred embodiments, the holding members 255 can be made of silicone, and in one or more embodiments, the holding members 255 can comprise a plurality of protrusions 260 on an inner wall of each holding member. The protrusions 260 can be of varying sizes on the inner wall of each holding member, and the protrusions are configured to capture portions of the medical cylindrical body (see FIG. 2H). For example, in instances in which the medical cylindrical body 265 is a guidewire, the wire can be captured between the two holding members 255 by the protrusions 260. In one or more embodiments, the protrusions 260 increase in size and density as one moves from the outside edge of the inner walls of each holding member 225 to the center of each holding member 255. Variable protrusion sizes allow for stronger or weaker holds on the cylindrical body (e.g., guidewires) depending on the diameter of the cylindrical body and how deep the operator presses the cylindrical body into the holder 200. While shown in the exemplary embodiments of FIGS. 2F-2H as circular, in one or more embodiments, the protrusions 206 can be of any geometrical shape, such as square, triangular, elliptical, or any mix thereof.

In one or more embodiments, the holding members 255 can be adhered to the respective inner surfaces of the outer members (e.g., via an adhesive). In at least one embodiment, the holding members 255 can rotate freely within the holder upon attachment of the connection members 220 and 230.

In one or more embodiments, the holding members 255 can be wetted with an antiseptic or antibacterial liquid such that upon pulling the medical cylindrical body (e.g., guidewire) through the holder 200, the medical cylindrical body glides along the surfaces of the holding members 255 thereby sterilizing or further cleaning the medical cylindrical body prior to insertion of the medical cylindrical body into the body. In one or more embodiments, the holding members 255 may also be wetted by the user with a sterile saline or water for cleaning or wetting of the medical cylindrical body.

The first and second outer members 205 and 210, the holding members 255, and the protrusions 260 can be sized and shaped to fit specific types of medical cylindrical body, and in particular, medical cylindrical bodies of varying sizes. For example, in the case of guidewires, the first and second outer members 205 and 210, holding members 255, and protrusions 260 can be respectively sized and shaped to securely hold guidewires of unique, different, or varying ranges of diameters.

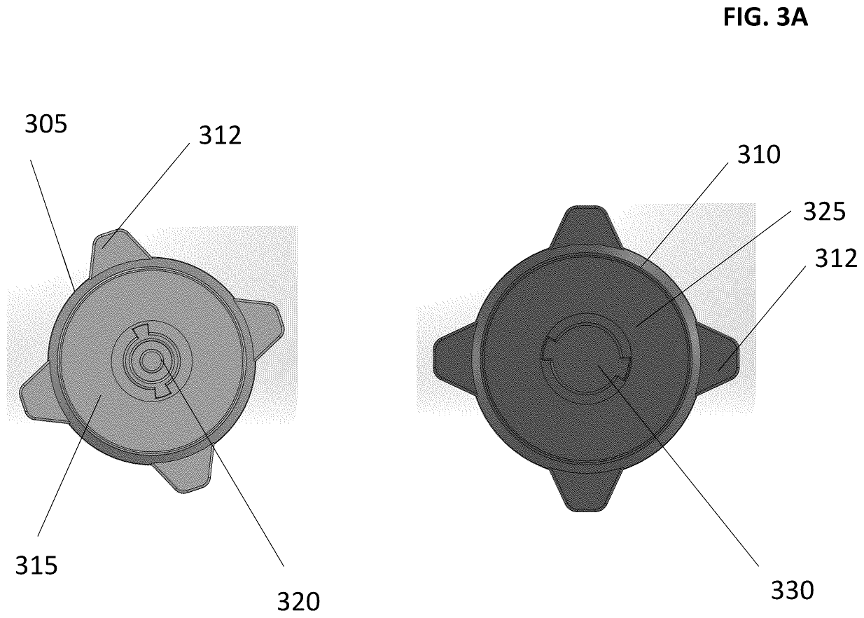

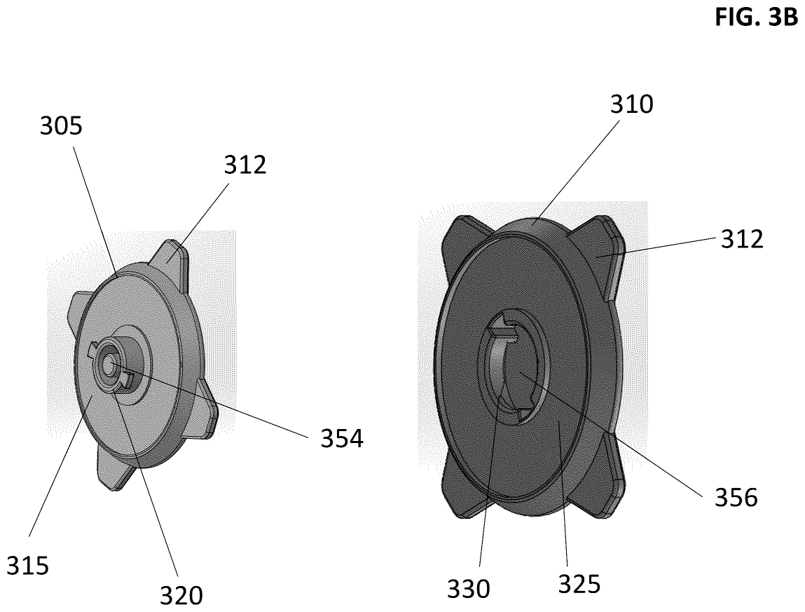

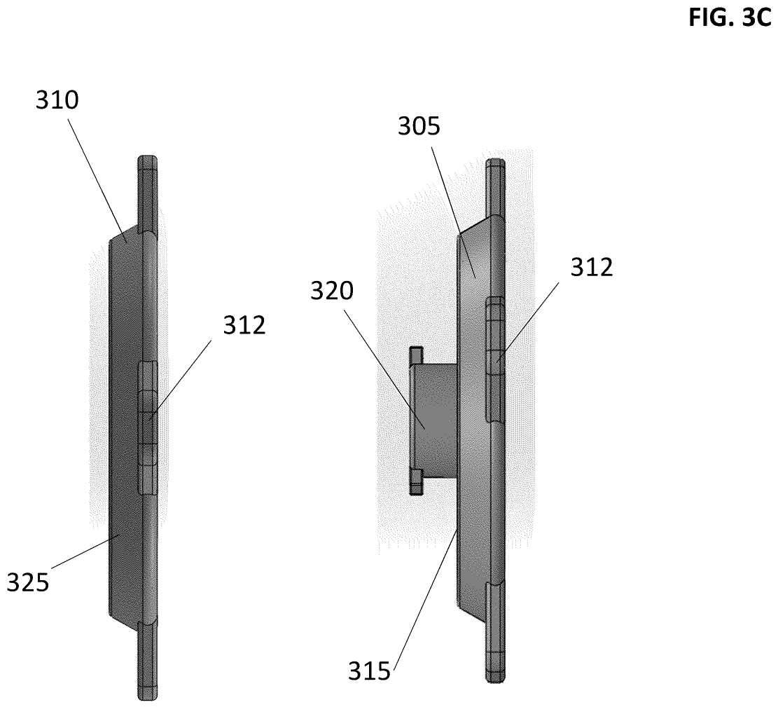

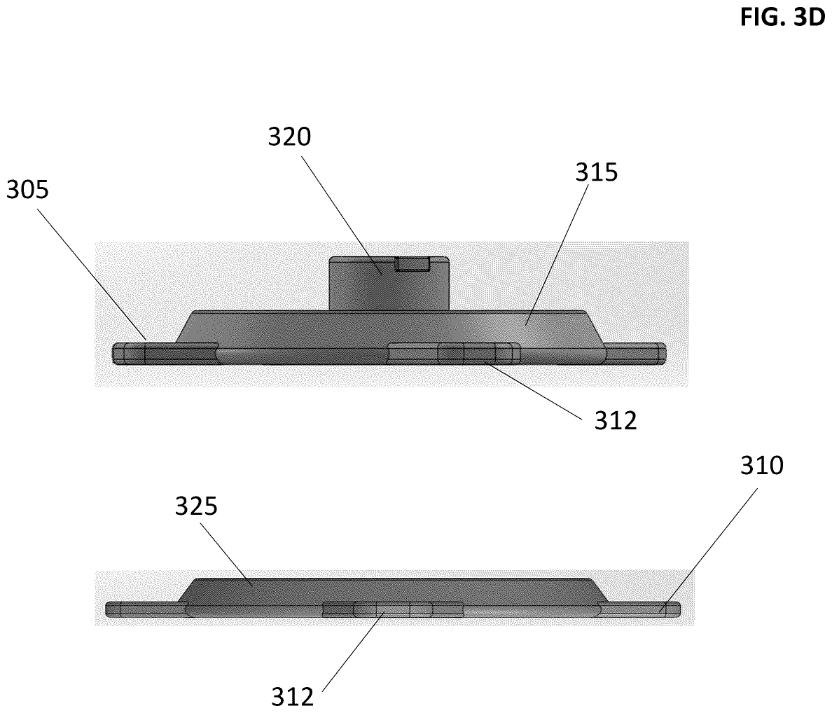

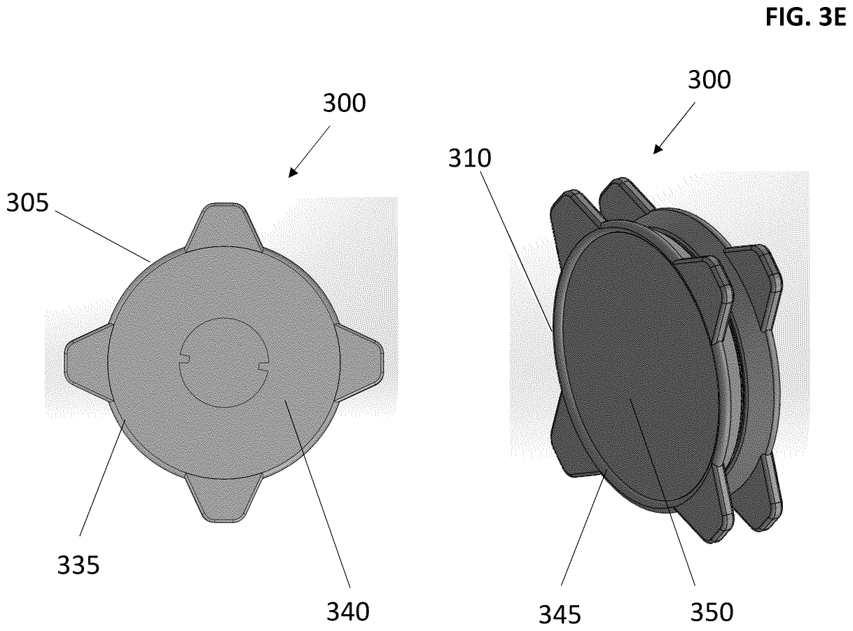

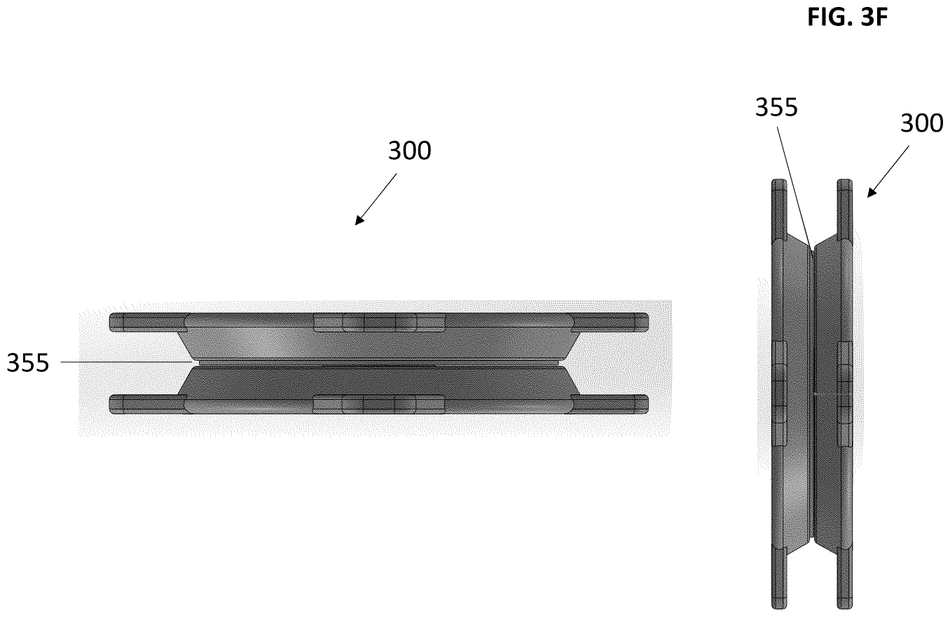

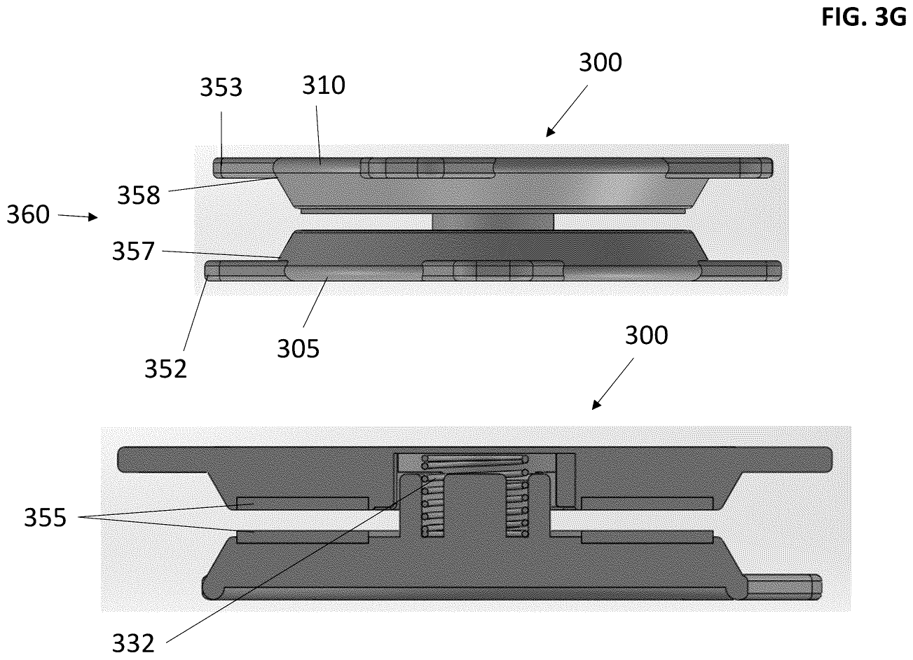

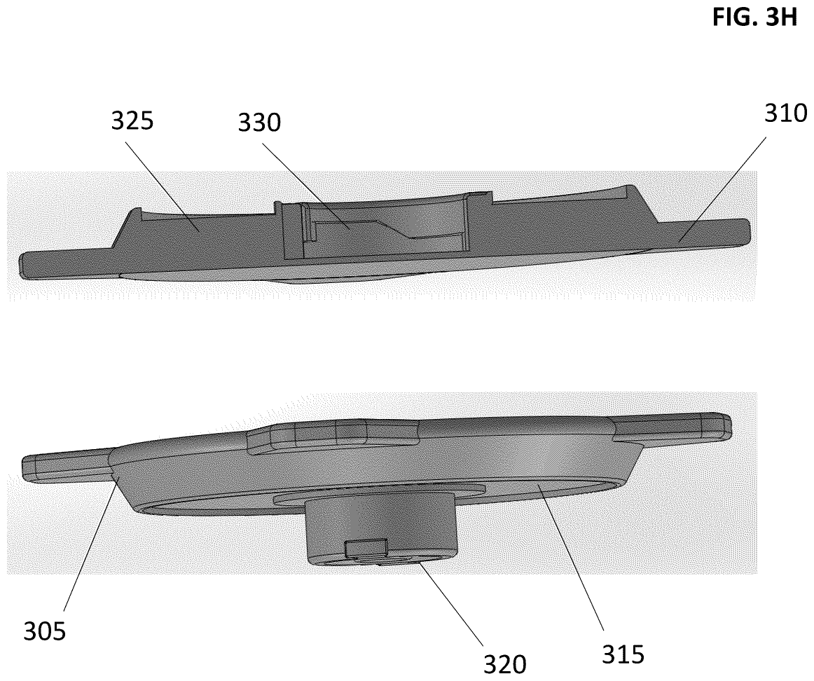

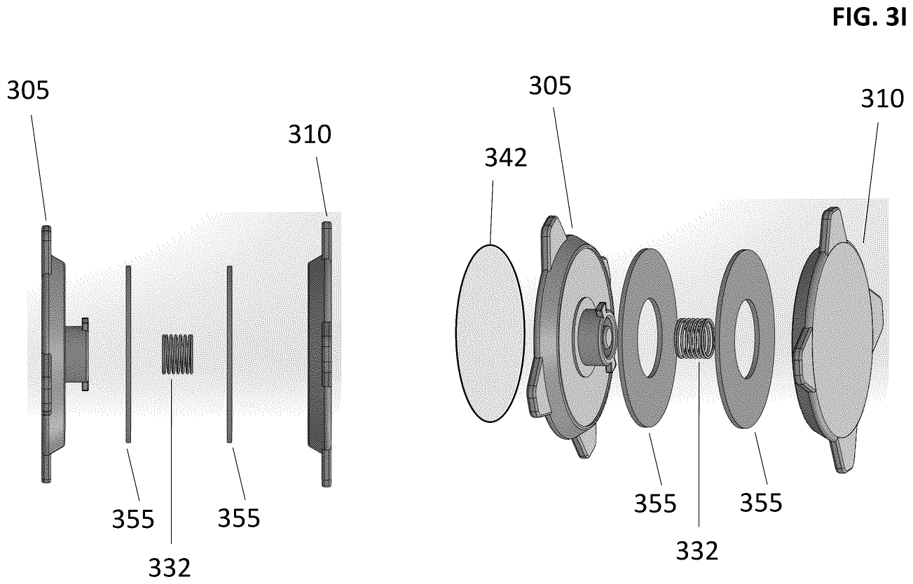

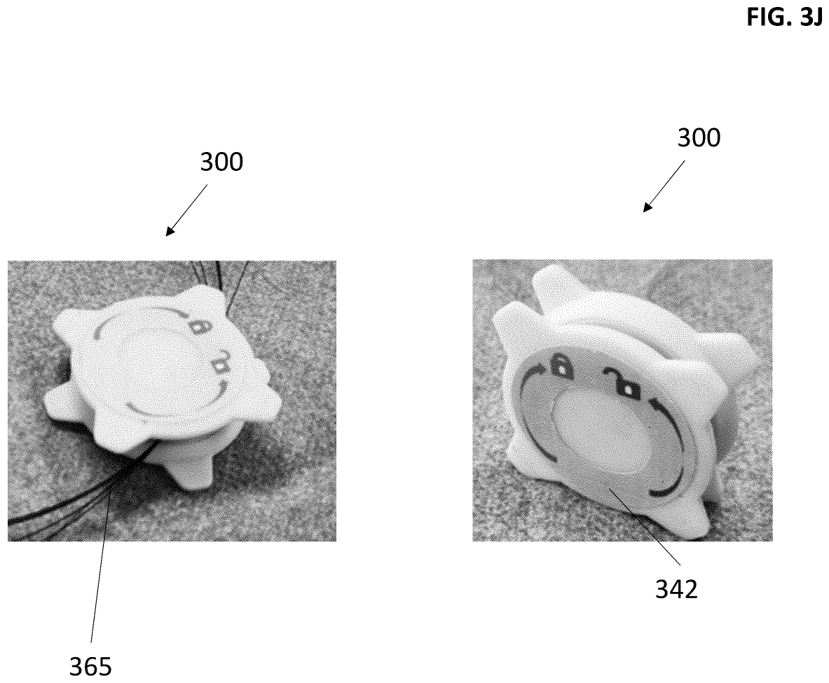

FIGS. 3A-3J disclose another embodiment of the holder for a medical cylindrical body of the present application, having similar components as the embodiments exemplified in FIGS. 1A-1H and FIGS. 2A-2H. With reference now to FIGS. 3A-3J, the holder 300 includes a first outer member 305 and a second outer member 310, which can be made of a plastic material or metal, or a similarly durable material. In one or more embodiments, each of the first and second outer members 305 and 310 can include a plurality of extensions 312 on their respective edges. The extensions 312 allow the holder 300 to balance or stand vertically on a flat surface, such as an operating table or a surface comprising a medical drape (see FIG. 3J, right image). These extensions 312 are also used to allow easier rotation or locking of the device, as discussed in further detail below. In one or more embodiments, each of the outer members 305 and 310 can include 2-4 extensions 312. In an exemplary embodiment, as shown in FIGS. 3A-3J, each of the outer members 305 and 310 can include 4 extensions 312. In at least one embodiment, the edges of the extensions 312 and the neighboring edges of the outer members 305 and 310 can include adhesive for attaching to a medical drape, such that the holder 300 can be stabilized in an upright position on the medical drape.

The first outer member 305 includes an inner surface 315 having a first connection member 320. As shown in FIG. 3B, in one or more embodiments, the first connection member 320 is a protruding member (“key”).

Similarly, the second outer member 310 also includes an inner surface 325 which includes a second connection member 330. In one or more embodiments, as shown in FIG. 3B, the second connection member 330 comprises a notch (“lock”) configured to securely receive the protruding member or key. The first outer member 305 and the second outer member 310 can be securely attached to one another in a locked position (closed position—FIG. 3F) upon insertion of the protruding member 320 into the notch 330 and subsequent turning of either the first or the second outer member in a first direction (e.g., clockwise). Similarly, after locking, in one or more embodiments, the first outer member 305 and the second outer member 310 can be unlocked by turning either the first or the second outer member in a second direction (e.g., counterclockwise) (unlocked position—FIG. 3G). In one or more embodiments, the locking (or unlocking) of the attachment between the first and second outer members 305 and 310 is accomplished via a ⅛^{th} turn of either the first or second outer member in the specified direction.

In one or more embodiments, the protruding member or key can comprise a compressible member 332 configured to assist in releasing the first outer member and the second outer member from the locked position upon turning either the first or second outer member in the second direction. The compressible member 332 is located in the center of the protruding member (first connection member 320) to allow for an assisted release from the locked to unlocked state when the holder is turned in the second direction (e.g., counter-clockwise). In one or more embodiments, the compressible member is a spring, as exemplified in FIGS. 3G and 3I.

With continued reference to FIGS. 3A-3I, the first outer member 305 also includes an outer surface 335 that optionally comprises a recess 340. The outer surface 335 or the recess 340 can be configured to receive an adhesive member 342. In one or more embodiments, the adhesive member 342 is a double-sided adhesive member, where one side attaches to the outer surface 335 or recess 340 and the other to the surface of an object (e.g., medical drape). In one or more embodiments, the adhesive member 342 is selectively detachable and re-attachable to the medical drape (e.g., a reusable medical drape or a disposable drape). The outer surface 335 or recess 340 is flat or substantially flat such that attaching it to the medical drape via the adhesive member 342 keeps the holder 300 attached as closely as possible and at a low profile relative to the medical drape.

Similarly, the second outer member 310 includes an outer surface 345 that optionally comprises a recess 350, where the outer surface 345 or the recess 350 is configured to be written on. Specifically, the outer surface 345 or the recess 350 can comprise a material that allows for writing via a marker, pen, or pencil, for example. The outer surface 345 or recess 350 can be a flat or substantially flat surface to allow for legible writing. In at least one alternative embodiment, instead of the outer surface 345 or recess 350 being configured to be written on, the outer surface 345 or recess 350 can be configured to receive a label that is of a material that allows for writing.

With specific reference to FIGS. 3I, in one or more embodiments, the holder 300 further includes two holding members 355 placed between the inner surface 315 of the first outer member 305 and the inner surface 325 of the second outer member 330. Each of the holding members 355 includes a throughhole for accommodating the first connection member 320 and/or the second connection member 330 upon attachment of the first and second outer members.

Upon attachment of the first and second outer members via their respective connection members 320 and 330, a space for holding the medical cylindrical body can be defined between the two holding members 355 or can be defined between the holding members 255 upon insertion of the medical cylindrical body. The holding members 355 are configured to hold the medical cylindrical body between them via tension when the first and second outer members are attached. The holding members 355 can be made of foam (e.g., plastic foam or open or closed cell plastic material), silicone, rubber or a similar deformable or flexible material that can securely hold the medical cylindrical body but without damaging the medical cylindrical body. In one or more preferred embodiments, the holding members 355 can be made of silicone.

In one or more embodiments, the holding members 355 can be wetted with an antiseptic or antibacterial liquid such that upon pulling the medical cylindrical body (e.g., guidewire) through the holder 300, the medical cylindrical body glides along the surfaces of the holding members 355 thereby sterilizing or further cleaning the medical cylindrical body prior to insertion of the medical cylindrical body into the body. In one or more embodiments, the holding members 355 may also be wetted by the user with a sterile saline or water for cleaning or wetting of the medical cylindrical body.

In one or more embodiments, the first and second outer members 305 and 310 and the holding members 355, can be sized and shaped to fit specific types of medical cylindrical body, and in particular, medical cylindrical bodies of varying sizes. For instance, in the case of guidewires, the first and second outer members 305 and 210, and the holding members 355 can be sized and shaped to securely hold guidewires of unique, different, or varying ranges of diameters.

As exemplified in FIGS. 3F-3G, both the first and second outer members 305 and 310 taper from an edge of the respective outer surfaces to the edge of the respective inner surfaces defining an annular funnel 360 configured to capture the medical cylindrical body. More specifically, as shown in FIG. 3G each of the first and second outer members 305 and 310 have respective outer perimeters 352 and 353, and respective centers 354 and 356 (see FIG. 3B) and respective surfaces 357 and 358 that taper from the outer perimeter 352, 353 to a point between the outer perimeter 352, 353 and the center 354, 356 defining the annular funnel 360 configured to capture the medical cylindrical body.

When the first outer member 305 and the second outer member 310 are attached to one another in an unlocked position (open position—FIG. 3G), the medical cylindrical body can be fed via the annular funnel 360 into the space between the holding member 355. When the first outer member 305 and the second outer member 310 are attached to one another in a locked position (closed position—FIG. 3F), the holding members 355 provide a more secure grip on the medical cylindrical body.

The funnel shape of the outer members also helps with the ability of the holder 300 to clean the medical cylindrical body. Specifically, as exemplified in FIGS. 3F-3G, the holding members of the holder 300 grip the cylindrical body 365 (e.g., guidewire) in a static state, but when force is applied by the operator, such as by pulling on the wire, the wire is able to pass through. As the wire passes through, the funnel shape of the holder 300 will naturally filter out large debris and small debris will be cleaned off as the wire passes through the holding members.

Accordingly, the holder of the present application can secure and release medical cylindrical bodies (e.g., guidewires, catheters) without direct user interaction with the holder. In other words, when the holder is attached to the surgical drape, the user can push the cylindrical body (e.g., wire) into it the holder (e.g. via the funnel) and the holder will hold the wire without the user having to physically open the outer members or even touch the holder. As such, the holder of the present application can hold and passively feed the cylindrical body during physician insertion into the patient.

In at least one alternative embodiment, the holder can be integrated into the surgical drape, thereby negating the need for an adhesive. In one or more embodiments, the holder can include an additional a flat surface attached to an edge of the first outer members. The flat surface can include an adhesive on its bottom surface for attachment to the surgical drape such that the holder stands vertically upon attachment of the flat surface to the drape, or for attachment to a table or area where the medical cylindrical bodies are being stored off the patient, when not in use.

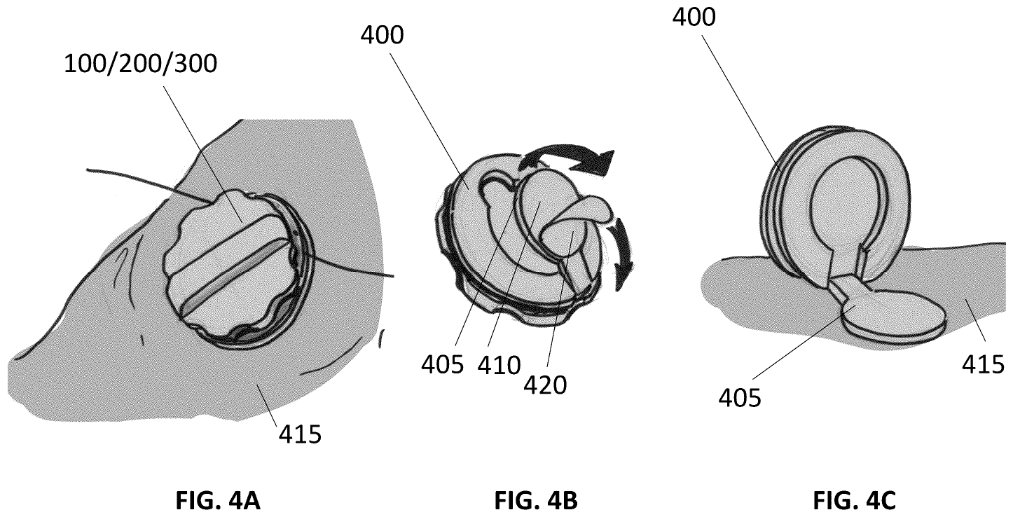

Exemplary embodiments of the attachment of the holder to the medical drape are shown in FIGS. 4A-4C. Specifically, FIG. 4A shows the typical attachment of the adhesive member on the outer surface or recess of the holder 100/200/300 as described herein, which allows the holder to adhere and lay flat against the medical drape. In at least one embodiment, as described above and shown in FIGS. 4B-4C, a holder 400 can include an additional flat surface 405 attached to an edge of the first outer members by a hinge or other articulating connection. It should be understood that the holder 400 can comprise substantially the same features as holder 100, 200, or 300 as described herein. The flat surface 405 of holder 400 can include an adhesive 410 such that when the flat surface 405 is pulled down into a horizontal position, e.g., extending normal to the outer surface of the holder, the adhesive 410 adheres to the drape 415. Alternately, flat surface 405 could be attached to a non-drape supporting structure, such as a table, patient gurney, or surgical tray for use proximate to a patient in surgery. In one embodiment, flat surface 405 may be hinged so that it will maintain a selected angle between 0 and 270 degrees relative to the outer surface of the holder, so as to maintain the holder in an appropriate orientation to the surface to which the flat surface 405 is adhered.

In one or more embodiments, the adhesive 410 can include a removable liner 420 for covering the adhesive 410. It should be understood the that adhesive 410 can comprise a sterilizable, double-sided, tackified acrylic adhesive, as discussed above with regard to other embodiments. It should also be understood that in one or more embodiments of the holder described herein, the adhesive or adhesive members can include a removable liner to prevent the adhesive from inadvertently attaching to a surface before the holder is in use.

In one or more embodiments, the adhesive can be substituted with one or more magnets (e.g. a pair of magnets). For example, the one or more magnets can be embedded in the outer member of the holder or adhered to the outer surface of the outer member. In such an embodiment, a magnetic base can be placed on or within the medical drape and the one or more magnets of the holder can selectively attach to magnetic base of the drape. As such, in this embodiment, the attachment of the holder to the drape is still selectively removable, but does not use an adhesive.

Also disclosed herein is a kit comprising one or more holders of the present application. In addition to the holder(s), in one or more embodiments, the kit can include one or more labels for attachment to an outer surface of one of the outer members, a writing utensil (e.g., marker) for writing on the labels, and a removeable covering for the adhesive on the outer surface(s) of one the outer members. In one or more embodiments, the kit can also include an instrument pad for wiping the cylindrical body. In at least one embodiment, the instrument pad can be an antiseptic instrument pad. In one or more embodiment, the kit can further include a card which allows for securing one or more holders in a package prior to use, or attachment to the adhesive of the holder for storage of the holder after it is removed from being attached to the drape. The kit can also include one or more medical cylindrical bodies, such as guidewires or catheters. The kit can also include cylindrical bodies of different diameters or lengths, for example. The kit is preferably provided in a sufficiently sterile packaging so that that it can be taken into and opened and then its contents used in a sterile patient operating environment without impairing the sterility of the operating theatre.

Also disclosed herein is a method for operating a holder for a medical cylindrical body. In accordance with one or more embodiments of the method, a holder of the present application is provided. Then, a portion of the medical cylindrical body is fed between the two holding members. The medical cylindrical body is then held between the two holding members of the holder via friction. In one or more embodiments of the method, the medical cylindrical body is a guidewire or a catheter or a surgical tool or an electrode for stimulating tissue. In at least one embodiment in which the cylindrical body is a guidewire, the method can further include the step of pulling the guidewire to cause a portion of the guidewire being held between the two holding members to be released, wherein the pulling of the guidewire causes the portion of the guidewire to be cleaned by the two holding members as it is released. In at least one embodiment, the method can further include a step of applying a cleaning liquid on the holding members.

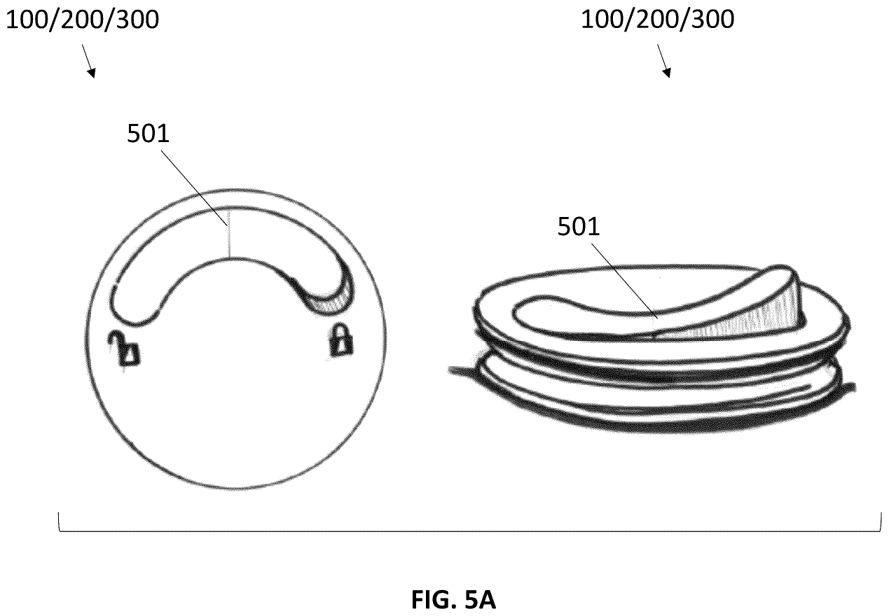

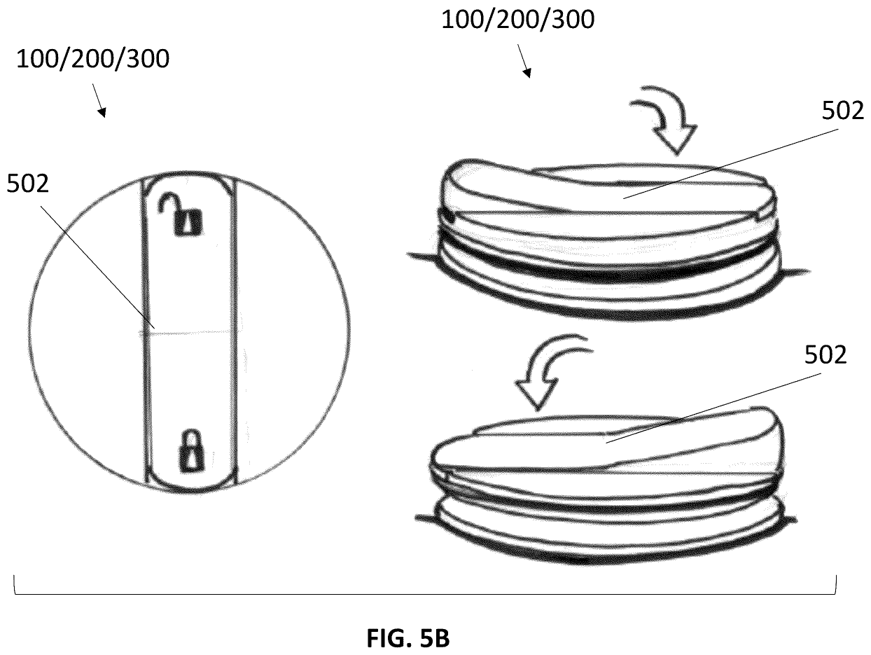

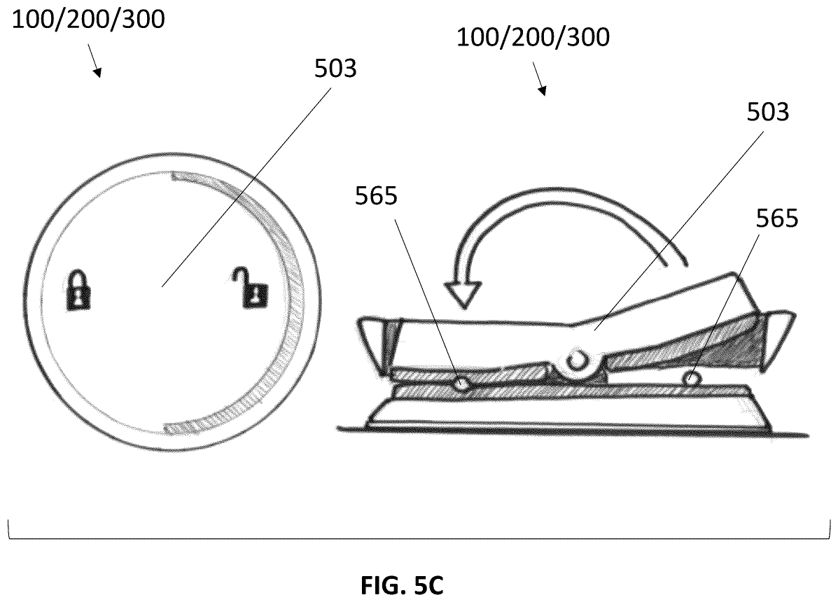

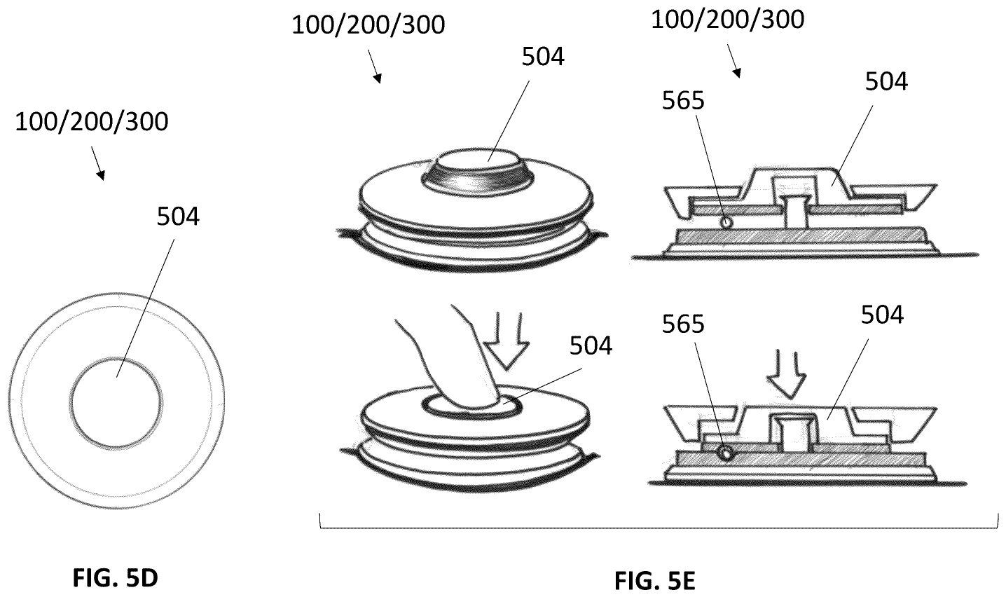

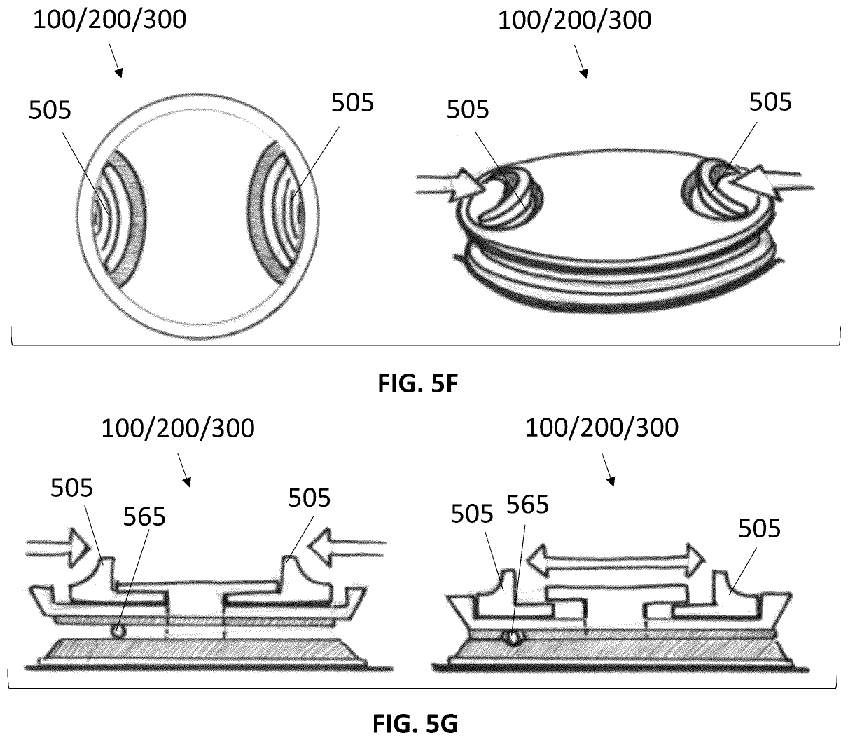

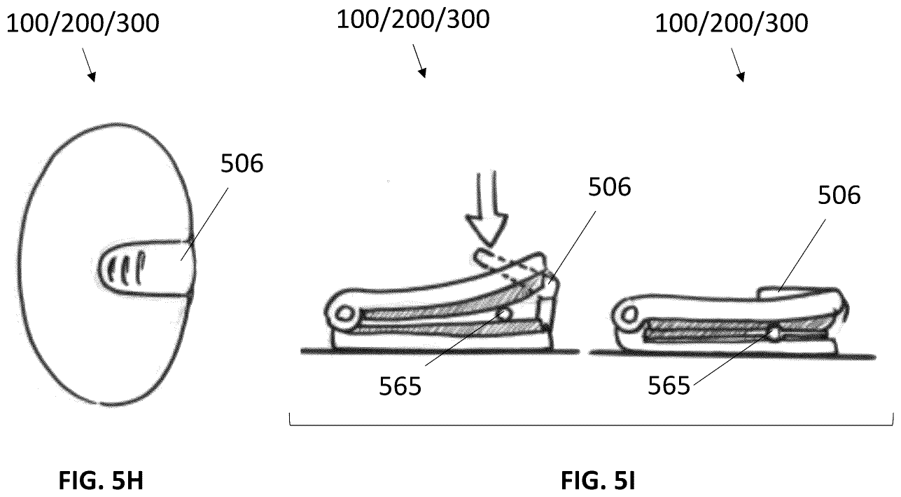

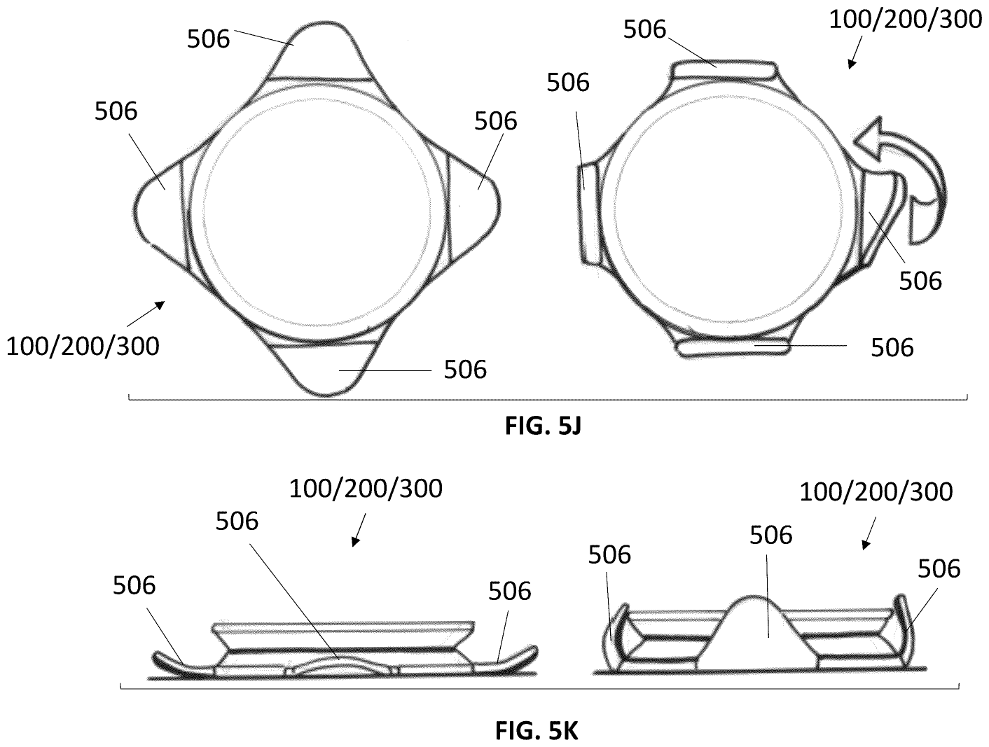

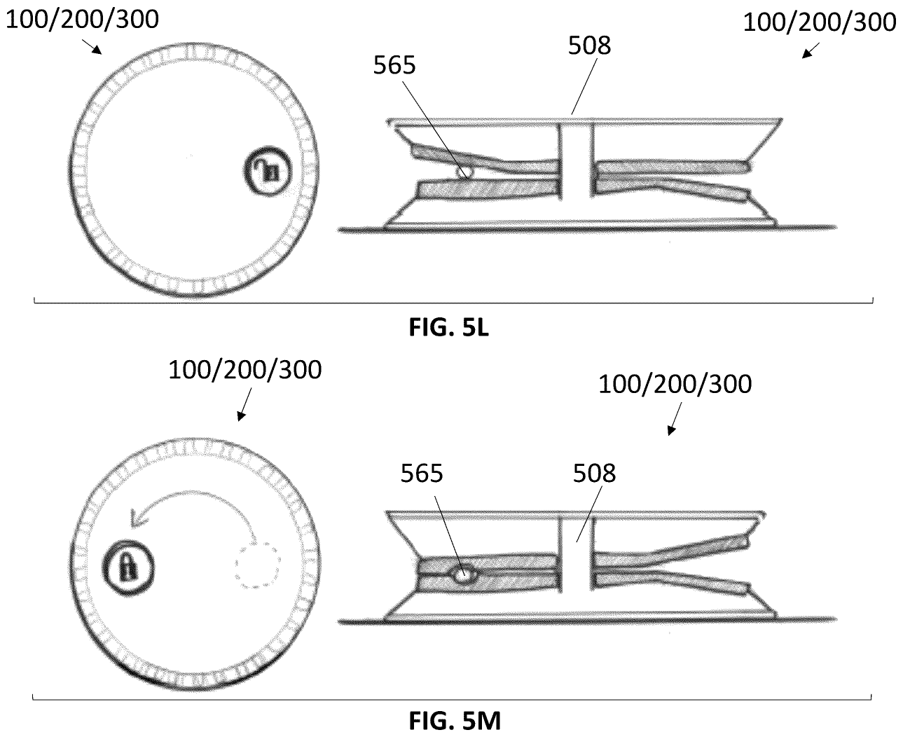

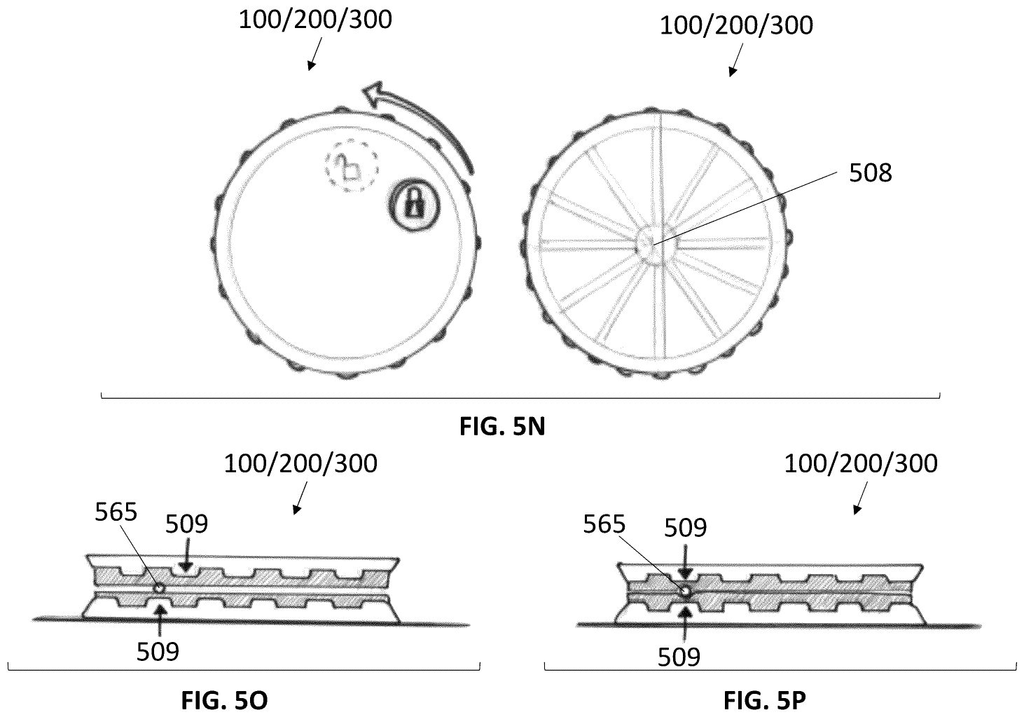

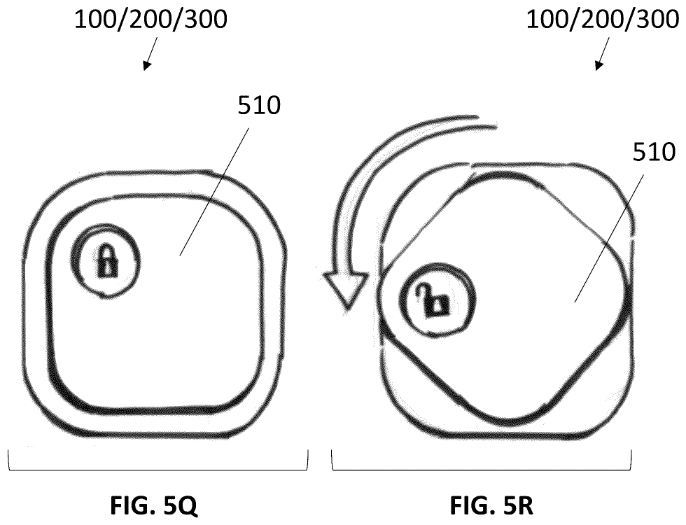

In one or more embodiments, the holder of the present application can further include a securing mechanism for securing the cylindrical body in the holder. This securing mechanism operates either independently or in combination with the holding members, as discussed previously, which hold the cylindrical body via friction. FIGS. 5A-5R display exemplary securing mechanisms in accordance with one or more embodiments. As shown in the FIGS. 5A-5R, in one or more embodiments, the security mechanisms further described below can be used for any of the holders of the present application (e.g., holders 100/200/300).

FIGS. 5A-5C display a top view (left) and a side view (right) of embodiments of the holder in which the second outer member of the holder (i.e., the outer member that does not attach to an adhesive) comprises a toggle button on its outer face wherein the toggle button (501, 502, 503) serves to selectively lock and unlock a clamping feature that secures the wire. Specifically, depression of one side of the toggle button configures a portion of the first outer member (top outer member) to apply additional pressure to the cylindrical body 565 (see FIG. 5C) secured within the holder, thereby providing further securing (clamping) the cylindrical body 565 between the two outer members. Likewise, depression of the other side of the toggle button configures the portion of the first outer member to release the pressure applied by the second outer member. As shown in FIGS. 5A-5C, the toggle button can be provided in a variety of different shapes with a “lock” and “unlock” portion on opposing sides, such as a crescent shape (FIG. 5A, 501), an oblong shape (FIG. 5B, 502), or a full-faced toggle button (FIG. 5C, 503) in which the “lock” portion of the button makes up all or substantially all of one side of the outer face of the first outer member, and the “unlock” portion of the button makes up all or substantially all of the other side of the outer face of the first outer member. Depression of the lock portion of the button 503 results in half of the device being clamped at a time, as exemplified shown in FIG. 5C.

Other exemplary securing mechanisms are shown in FIGS. 5D-5Q. Specifically, FIGS. 5D-5E display a securing mechanism having a “pop” button 504 integrated into the first outer member (top outer member) in accordance with one or embodiments. As shown in FIGS. 5E, when the button 504 is depressed by the user, the button 504 stays in a down position (lock position) and applies pressure to the cylindrical body 565. Once in the down position, if the button 504 is pressed again, the button 504 is released back to an up position, and the applied pressure on the cylindrical body is released.

FIGS. 5F-5G display a securing mechanism having a squeeze locking mechanism in accordance with one or more embodiments. In this embodiment as exemplified in FIG. 5F, the securing mechanism comprises two squeeze buttons 505 on opposing ends of the first outer member of the holder (i.e., the top outer member that does not comprise an adhesive). In this embodiment, the holder remains in the locked position (i.e., pressure is being applied to the cylindrical body held in the holder) until the user squeezes the two buttons 505 together toward the middle of the first outer member (i.e., using two fingers). The squeezing of the buttons 505 together releases the pressure applied by the first outer member on the cylindrical body 565 as shown in FIG. 5G.

FIGS. 5H-5I display a securing mechanism that includes a latch 506 (e.g., hinged latch) for latching an end of the first outer member to an end of the second outer member, thereby pressing the two outer members tightly together to apply pressure to the cylindrical body 565. The latch 506 can be attached to an end of the second outer member (bottom outer member) and can have an opening through which a portion of an end of the first outer member fits through when the latch 506 is engaged.

FIGS. 5J-5K display a securing mechanism that includes a plurality of locking tabs 507 on the edges of the second outer member (bottom outer member). Instead of locking by squeezing the first and second outer members together on the cylindrical body as done in the previously discussed securing mechanisms, the locking tabs 507 prevent the cylindrical body from sliding out of the holder by folding upwards (see FIGS. 5J-5K, right panel). In one or more embodiments, there are multiple tabs 507 to allow for different types of placements for the cylindrical body. In the exemplary embodiment of FIGS. 5J-5K, there are four tabs 507; however, in other embodiments, there can be a different number of tabs, such as 2 or 3 or 5 or 6. In one or more implementations, not all of the tabs 507 need be used at once, only the tabs that close the cylindrical body in the holder. For example, when a cylindrical body is held on one side of the holder, only 2 or 3 tabs may need to be folded upwards to hold the cylindrical body in position and prevent it from sliding out of the holder.

FIGS. 5L-5M display a securing mechanism in which the first outer member (top outer member can be rotated to lock (apply additional pressure to the cylindrical body) or unlock (release additional pressure on the cylindrical body) the holder. In this embodiment, the first outer member (top outer member) and the second outer member (bottom outer member) of the holder are not uniformly shaped in that one end of each outer member tapers toward the outside edge while the other end of each outer member does not taper and remains substantially flat. The first outer member is configured to selectively rotated approximately 180 degrees about a central axis 508 between a locked and unlocked position, wherein in the locked position, the cylindrical body is held between the matching portions of the outer members that do not taper, such that additional pressure is applied to the cylindrical body 565 (see FIG. 5M). When the first outer member is rotated to the unlocked position, the cylindrical body 565 is then held between the tapered portion of first outer member and the flat portion of the second outer member (see FIG. 5L).

FIGS. 5N-5P display another rotating securing mechanism. In this embodiment, each of the first and second outer members include gear-like protrusions 509 at regular (or irregular) intervals along their respective inner surfaces. As the first outer member (top outer member) is rotated a specified amount (e.g., 30 degrees) around a central axis 508, the protrusions 509 of the first and second outer members are either aligned with one another (i.e., on top of one another) or misaligned with one another. When the first outer member is rotated such that the cylindrical body 565 is held at a location in which the protrusions 509 of both the first and second outer members are aligned (see FIG. 5P), additional pressure is applied the cylindrical body 565 to secure the cylindrical body in the holder (locked position). Conversely, when the first outer member is rotated such that the cylindrical body 565 is held at a location in which the protrusion 509 of both the second outer member (bottom outer member) is not aligned with a protrusion 509 of the first outer member (see FIG. 5O), less pressure is applied to the cylindrical body 565. This embodiment, as exemplified in FIGS. 5N-5P allows for a shorter turn radius or rotation (e.g., 30 degrees) for moving the holder into locked position as compared with the embodiment of 5L-5M, which utilizes approximately a 180-degree rotation for locking.

FIGS. 5Q-5R display an alternative to the embodiments of FIGS. 5L-5M or 5N-5P. Specifically, the embodiments exemplified by FIGS. 5Q and 5R can operate in the same fashion as FIGS. 5L-5M (with the tapering outside edges) or FIGS. 5N-5P (with aligning protrusions on the inner surfaces of the outer members), with the exception that the first and second outer members 510 are generally rectangular—or more preferably square-shaped instead of circular. In this embodiment, the first outer member 510 (top outer member) is rotated at specified amounts (e.g. 90 degrees) around the central axis to apply additional pressure (lock) to secure the cylindrical body in the holder or to release the additional pressure (unlock). For example, as exemplified in FIGS. 5Q-5R, when unlocked, the first outer member (top outer member) is at an angle in relation to the second outer member (bottom outer member) (see FIG. 5R), but when in the locked position, the square edges of the outer members visually align (e.g., are parallel) (see FIG. 5Q). Accordingly, this embodiment provides a more visible indication of when the device is locked or unlocked.

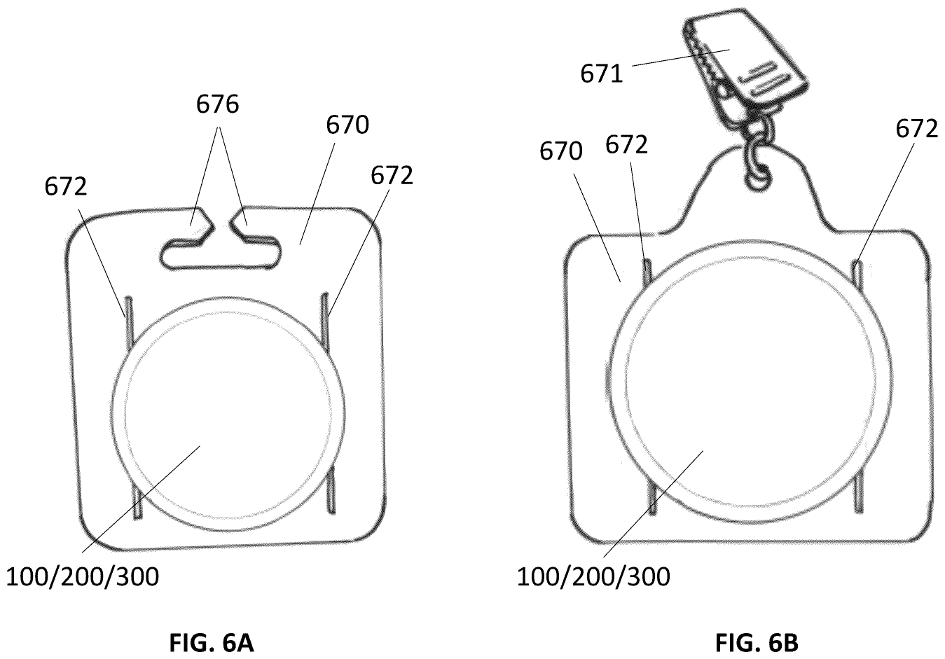

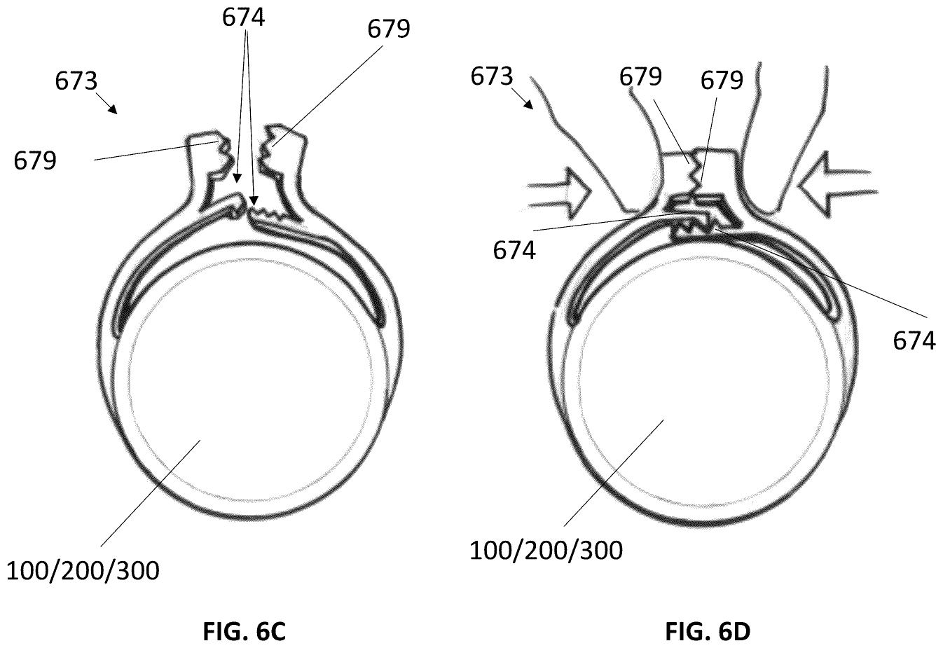

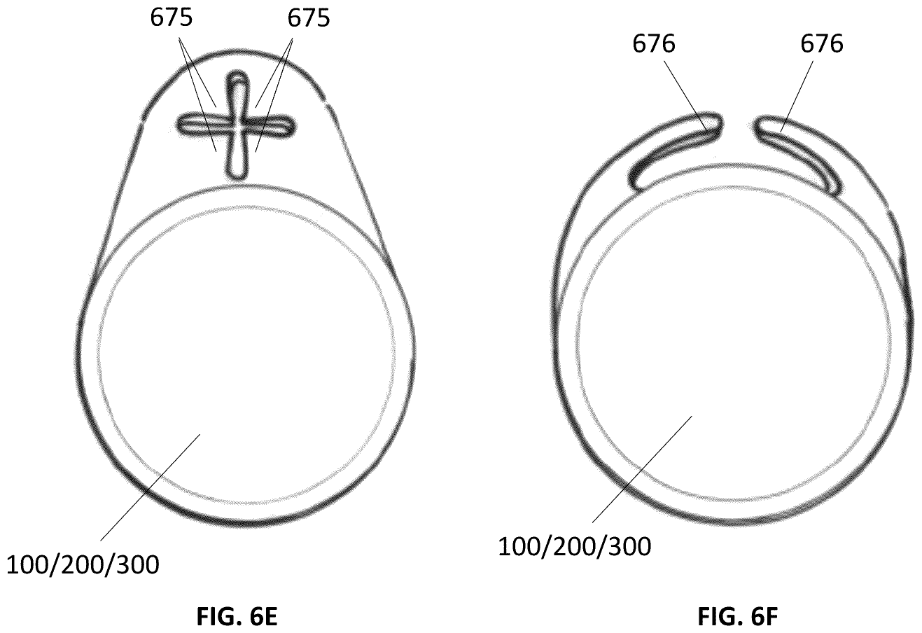

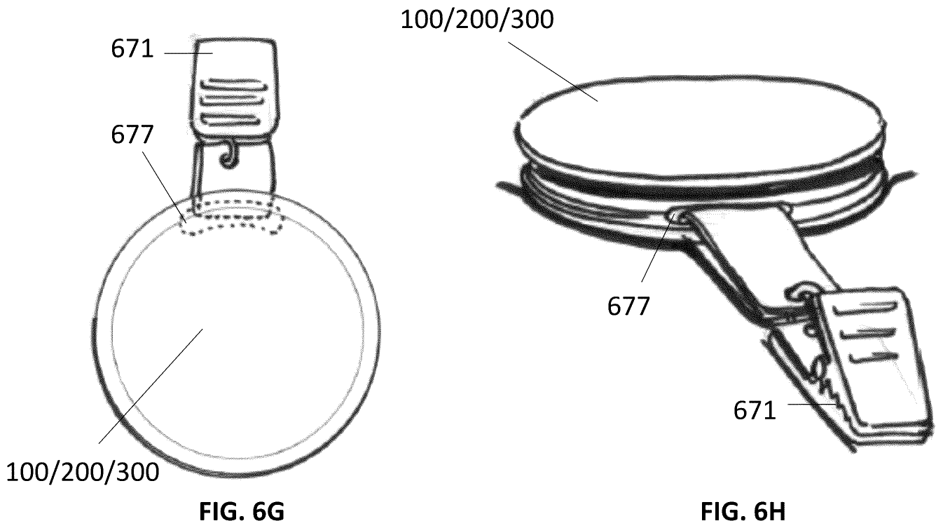

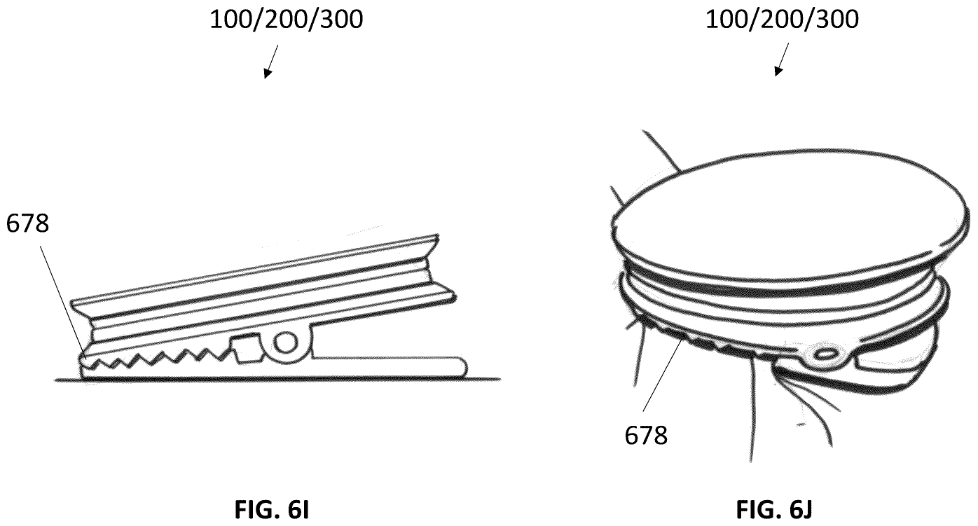

In one or more embodiments, the holder of the present application can further include a secondary attachment mechanism by which the holder of the present application can be secured to a surgical drape or a user's garment. The secondary attachment mechanism can attach to (e.g., integrated into) either the first outer member or the second outer member, wherein the secondary attachment mechanism is configured to attach to a drape or a garment. The secondary attachment mechanism can operate either independently or in combination with an additional method such as the adhesive on the second outer member (bottom outer member). FIGS. 6A-6J display exemplary secondary attachment mechanisms in accordance with one or more embodiments. As shown in the FIGS. 6A-6J, in one or more embodiments, the secondary attachment mechanisms further described below can be used for any of the holders of the present application (e.g., holders 100/200/300).

Advantageously, it has been discovered that use of a secondary attachment mechanism allows the medical worker to use the holder as a third hand during use of the medical cylindrical body. For example, by attaching the holder to the medical worker's surgical clothing (i.e., garment), at least a portion of the medical cylindrical body can be secured by the holder as the worker is working with the medical cylindrical body, and then the worker can detach the holder from the garment and reattach it elsewhere, e.g., to the surgical drape, or a table, or tray for securing the cylindrical medical body as needed, leaving the medical worker free to move away from the patient and stored cylindrical medical body.

FIGS. 6A-6B display an embodiment in which the holder of the present application is attached to a pouch card 670 having a secondary attachment mechanism 671. The pouch card 670 is used to stabilize the holder in its sterilizable packaging. The pouch card can include a pair of slits 672 such that the holder can removably attach to the pouch card 670 by tucking opposing edges of one of the outer members into the slits 672. The pouch card 670 can include a secondary attachment mechanism 671 for attaching to a drape or garment, such as an attached alligator clip (see FIG. 6B) or teeth/projections that grip in the manner of a bread tie, for example, (see FIG. 6A). In one or more embodiments, the pouch card 670 and secondary attachment mechanism 671 can be additional parts of a kit comprising the holder.

FIGS. 6C-6D display an embodiment in which the holder of the present application includes a secondary attachment mechanism in the form of an integrated ratchet clip 673. As exemplified in FIGS. 6C and 6D, in one or more embodiments the ratchet clip 673 can be molded in one piece with one of the outer members of the holder. As shown in FIG. 6D, when pressed together, the opposing ends 674 of the clip 673 can ratchet together to provide varying degrees of tightness to urge protrusions 679 sufficiently tightly together about a fold of drape or garment to secure the holder thereto. In at least one alternative embodiment, the ends 674 of the ratchet mechanism only have one point of attachment, and thus one degree of tightness.

FIGS. 6E-6F display embodiments in which the holder of the present application include one or more integrated teeth as a secondary attachment mechanism. The integrated teeth can be used to grip a fold of drape or garment and can be molded into one of the outer members of the holder of the present application. For example, as shown in the embodiment of FIG. 6E, a portion of the drape or garment can be pushed through the four integrated teeth 675. Similarly, in the embodiment of FIG. 6F (and FIG. 6A), a portion of the drape or garment can be pushed into and held within the opening and the two integrated teeth 676 in the manner of a bread tie.

Similarly, FIGS. 6G-6H display an embodiment in which the holder of the present application can include a hole 677 molded into one of the outer members of the holder through which a secondary attachment mechanism 671 can be attached, such as a clip (e.g., alligator clip), for attaching to a drape or garment.

FIGS. 6I-6J display another embodiment in which the holder of the present application includes a spring-action clip 678 that is attached to one of the outer members of the holder. The spring-action clip 678 can be used to attach to a portion of a drape or garment as shown in FIG. 6J. In one or more embodiments, the spring-action clip 678 can be molded into one of the outer members of the holder.

Specific embodiments of the present application are further described in the items below.
Item 1. A holder for an elongated medical body suitable for insertion into a patient during a medical procedure, comprising:
   a first outer member having a first perimeter, a first inner surface and a first outer surface, at least a portion of the first outer member lying in a first plane;
   a second outer member having a second perimeter, a second inner surface and a second outer surface, at least a portion of the second outer member lying in a second plane;
   a core extending between the first and second outer members at a location interior to the first and second perimeters, wherein the first and second outer members are maintained with the first and second inner surfaces in opposition about the core with the first and second planes oriented generally in parallel and spaced apart; and
   a compressible material disposed on at least a portion of one of the first and second inner surfaces;
   wherein the first and second inner surfaces are configured to engage and hold the elongated medical body when the elongated medical body is advanced therebetween towards the core and is in engagement with the compressible material.
Item 2. The holder of item 1, further comprising:
   a writeable surface disposed on the outer surface of one of the first and second outer members.
Item 3. The holder of item 1 or item 2, further comprising:
   an adhesive disposed on one of the outer surfaces of the first and second outer members
   wherein the adhesive is configured to attach to one of the first and second outer surfaces and to a surface not forming part of the holder.
Item 4. The holder of one or items 1-3, wherein the core further comprises a first connector attached to one of the first and second outer members, wherein the first connector is connected to the other of the first and second outer members.
Item 5. The holder of one of items 1-5, wherein the first and second inner surfaces are configured to form a funnel cross sectional shape, for example an annular funnel.
Item 6. A holder for an elongated medical body suitable for insertion into a patient during a medical procedure, comprising:
   a first outer member having a first inner surface, and a first outer surface,
   a second outer member having a second inner surface and a second outer surface,
   a core structure extending between the first and second outer members;
   an adhesive member configured to adhere the holder to a first surface;
   two holding members respectively placed between the inner surfaces of the first and second outer members, and wherein the holding members are compressible and configured to hold therebetween the elongated medical body; and
   wherein the adhesive member is configured to attach to one of the first and second outer surfaces.
Item 7. The holder of item 6, wherein the core structure comprises a first connection member coupled to the first outer member and a second connection member coupled to the second outer member.
Item 8. The holder of item 6 or item 7, further comprising a writable surface on the first and second outer surfaces.
Item 9. The holder of one of items 6-8, wherein each of the first and second outer members have an outer perimeter and a center and a surface that tapers from the outer perimeter to a point between the outer perimeter and the center defining a funnel configured to capture the elongated medical body.
Item 10. The holder of one of items 6-9, wherein the attachment of the first and second outer members defines a space suitable for holding the elongated medical body between the two holding members.
Item 11. The holder of one of items 6-10, wherein two holding members further comprise a foam material.
Item 12. The holder of one of items 6-10, wherein the two holding members further comprise a silicone material.
Item 13. The holder of item 12, wherein at least one of the holding members further comprise a plurality of protrusions, wherein the protrusions are configured to capture portions of the elongated medical body.
Item 14. The holder of item 13, wherein at least some of the protrusions are of differing sizes.
Item 15. The holder of one of items 6-14, wherein at least one of the first and second outer members includes a recess on its outer surface relative to its outer edge, wherein the recess comprises a recess surface configured to receive labeling or the adhesive member.
Item 16. The holder of one of items 6-15, wherein the first connection member comprises two prongs and the second connection member comprises two openings, wherein the two prongs are configured to securely fit into and attach to the two openings.
Item 17. The holder of one of items 6-15, wherein the first connection member comprises a protruding member and the second connection member comprises a notch configured to securely receive the protruding member, wherein the first outer member and the second outer member are securely attached to one another having an axis of rotation, and in a locked position upon insertion of the protruding member into the notch and subsequent turning of either the first or the second outer member in a first direction about the axis of rotation.
Item 18. The holder of item 17, wherein the protruding member comprises a compressible member configured to assist in releasing the first outer member and the second outer member from the locked position upon turning either the first or the second outer member in a second direction about the axis of rotation.
Item 19. The holder of item 18, wherein the compressible member is a spring.
Item 20. The holder of one of items 6-19, wherein the writable surface comprises a label adhered to the outer surface of the respective outer member.
Item 21. The holder of one of items 6-20, wherein the adhesive member maintains its adhesive qualities upon multiple attachments and detachments from the first surface.
Item 22. The holder of one of items 6-21, wherein the adhesive member is configured to removably attach to the first surface, and wherein the first surface is a drape, a garment, a table, or surgical tray.
Item 23. The holder of one of items 6-22, further comprising a toggle button integrated into the first outer member, wherein depression of one side of the toggle button configures a portion of the first outer member to apply additional pressure to the elongated medical body when in it is held within the holder, thereby further securing the elongated medical body within the holder, and wherein depression of the other side of the toggle button configures the portion of the first outer member to release the additional pressure.
Item 24. The holder of one of items 6-22, further comprising a button integrated into the first outer member and configured to be selectively depressed into the first outer member, wherein depression of the button applies additional pressure to the elongated medical body when it is held within the holder.
Item 25. The holder of one of items 6-22, further comprising two buttons integrated into opposing ends of the first outer member, wherein when the two buttons are in a static position, the first outer member is configured to apply additional pressure to the elongated medical body when in it is held within the holder, and wherein when the two buttons are squeezed together toward the middle of the first outer member, the first outer member is configured to release the pressure applied by the first outer member on the elongated medical body.
Item 26. The holder of one of items 6-22, further comprising a latch attached to an end of the second outer member, wherein the latch is configured to engage with an end of the first outer member, and wherein the latch includes an opening through which the end of the first outer member is configured to fit within when the latch is engaged.
Item 27. The holder of one of items 6-22, further comprising a plurality of locking tabs located on the edge of the second outer member, wherein the locking tabs are configured to selectively fold upwards to prevent the elongated medical body from sliding out of the holder when the elongated medical body is held within the holder.
Item 28. The holder of one of items 6-22, further comprising a central axis between the first outer member and the second outer member, wherein each of the first and second outer member comprises one end that tapers towards the outside edge of the respective outer member and another end that does not taper toward the outside edge, and wherein the first outer member is configured to selectively rotated approximately 180 degrees about a central axis such that the non-tapered ends of the first and second outer members are aligned, thereby applying pressure to the elongated medical body held between the non-tapered ends.
Item 29. The holder of one of items 6-22, further comprising a central axis between the first outer member and the second outer member, wherein each of the first and second outer members include gear-like protrusions at intervals along their respective inner surfaces, and wherein the first outer member is configured to selectively rotated at specified intervals around the central axis, such when the protrusions of both the first and second outer members are aligned, additional pressure is applied to the elongated medical body when it is held between the respective protrusions.
Item 30. The holder of item 29, wherein the first and second outer members are substantially square-shaped.
Item 31. The holder of one of items 6-22, further comprising a central axis between the first outer member and the second outer member, wherein the first and second outer members are substantially square-shaped, wherein each of the first and second outer members comprises one end that tapers towards the outside edge of the respective outer member and another end that does not taper toward the outside edge, and wherein the first outer member is configured to selectively rotate approximately 90 degrees about the central axis such that that the non-tapered ends of the first and second outer members are visually aligned, thereby applying pressure to the elongated medical body held between the non-tapered ends.
Item 32. The holder of one of items 6-31, further comprising a ratchet clip attached to either the first outer member or the second outer member, wherein the ratchet clip comprises opposing ends configured to selectively attach to provide one or more degrees of tightness, and wherein the ratchet clip is configured to attach to a drape or a garment.
Item 33. The holder of one of items 6-31, further comprising a plurality of integrated teeth molded into the first outer member or the second outer member, wherein the integrated teeth configured to attach to a drape or a garment.
Item 34. The holder of one of items 6-31, further comprising a clip for configure to attach to a drape or a garment, wherein the clip is removably attachable to the holder via a hole molded into the first outer member or the second outer member.
Item 35. The holder of one of items 6-31, further comprising a spring-action clip attached to the first outer member or second outer member, wherein the spring-action clip is configured to attach to a portion of a drape or a garment.
Item 36. The holder of one of items 6-31, further comprising a secondary attachment mechanism attached to either the first outer member or the second outer member, wherein the secondary attachment mechanism is configured to attach to a drape or a garment.
Item 37. A method for operating a holder for an elongated medical body, the method comprising:
   providing the holder, wherein the holder comprises:
      a first outer member having a first inner surface and a first outer surface,
      a second outer member having a second inner surface and a second outer surface,
      a core structure extending between the first and second outer members;
      an adhesive member;
      two holding members respectively placed between the inner surfaces of the first and second outer members,
      wherein each of the first and second outer members have an outer perimeter and a surface that tapers from the outer perimeter to a point on the respective inner surface defining a funnel configured to capture the elongated medical body;
   feeding a portion of the elongated medical body, via the funnel, between the two holding members; and
   holding the elongated medical body between the two holding members via friction.
Item 38. The method of item 37, wherein at least one of the first and second outer members includes a recess on its outer surface relative to its outer edge, wherein the recess is configured to receive a label having a writable surface or the adhesive member.
Item 39. The method of item 37 or item 38, further comprising:
   attaching the adhesive member to a first surface.
Item 40. The method of item 39, wherein the adhesive member is configured to removably attach to the first surface, and wherein the first surface is a drape, a garment, a table, or surgical tray.
Item 41. The method of one of items 37-40, wherein the elongated medical body is a guidewire.
Item 42. The method of item 41, further comprising:
   pulling the guidewire to cause a portion of the guidewire held between the two holding members to be released, wherein the pulling of the guidewire causes the portion of the guidewire to be cleaned by the two holding members as it is released.
Item 43. The method of item 42, further comprising:
   applying a cleaning liquid on the holding members.
Item 44. A kit comprising:
   the holder of one of items 1-36;
   a writing utensil configured to write on a writable surface of the holder, and a removeable covering for the adhesive member of the holder; and
   an instrument pad for wiping an elongated medical body.
Item 45. The kit of item 44, wherein the instrument pad is an antiseptic instrument pad.
Item 46. The kit of item 44 or item 45, further comprising at least one elongated medical body, wherein the at least one elongated medical body is a guidewire or a catheter.
Item 47. The kit of one of items 44-46, further comprising a pouch card configured for selective attachment to the holder of one of items 1-36, wherein the pouch card is removably fastened to an attachment mechanism.

Although much of the foregoing description has been directed to holders for a medical cylindrical body or methods for operating a holder for a medical cylindrical body or kits comprising one or more holders, the apparatuses and methods disclosed herein can be similarly deployed and/or implemented in scenarios, situations, and settings far beyond the referenced scenarios. It should be further understood that any such implementation and/or deployment is within the scope of the apparatuses and methods described herein.

It is to be further understood that like numerals in the drawings represent like elements through the several figures, and that not all components and/or steps described and illustrated with reference to the figures are required for all embodiments or arrangements. Further, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “including,” “comprising,” or “having,” “containing,” “involving,” and variations thereof herein, when used in this specification, specify the presence of stated features, integers, steps, operations, elements, and/or components, but do not preclude the presence or addition of one or more other features, integers, steps, operations, elements, components, and/or groups thereof.

It should be noted that use of ordinal terms such as “first,” “second,” “third,” etc., in the application to modify an element does not by itself connote any priority, precedence, or order of one element over another or the temporal order in which acts of a method are performed, but are used merely as labels to distinguish one element having a certain name from another element having a same name (but for use of the ordinal term) to distinguish the elements.

Notably, the figures and examples above are not meant to limit the scope of the present disclosure to a single implementation, as other implementations are possible by way of interchange of some or all the described or illustrated elements. Moreover, where certain elements of the present disclosure can be partially or fully implemented using known components, only those portions of such known components that are necessary for an understanding of the present disclosure are described, and detailed descriptions of other portions of such known components are omitted so as not to obscure the disclosure. In the present specification, an implementation showing a singular component should not necessarily be limited to other implementations including a plurality of the same component, and vice-versa, unless explicitly stated otherwise herein. Moreover, applicants do not intend for any term in the application to be ascribed an uncommon or special meaning unless explicitly set forth as such. Further, the present disclosure encompasses present and future known equivalents to the known components referred to herein by way of illustration.

The foregoing description of the specific implementations will so fully reveal the general nature of the disclosure that others can, by applying knowledge within the skill of the relevant art(s), readily modify and/or adapt for various applications such specific implementations, without undue experimentation, without departing from the general concept of the present disclosure. Such adaptations and modifications are therefore intended to be within the meaning and range of equivalents of the disclosed implementations, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s). It is to be understood that dimensions discussed or shown are drawings are shown accordingly to one example and other dimensions can be used without departing from the disclosure.

The subject matter described above is provided by way of illustration only and should not be construed as limiting. Various modifications and changes can be made to the subject matter described herein without following the example embodiments and applications illustrated and described, and without departing from the true spirit and scope of the invention encompassed by the present disclosure and equivalent structures and functions or steps.

What is claimed is:

1. A holder for an elongated medical body suitable for insertion into a patient during a medical procedure, comprising:
a first outer member having a first perimeter, a first inner surface and a first outer surface, at least a portion of the first outer member lying in a first plane;
a second outer member having a second perimeter, a second inner surface and a second outer surface, at least a portion of the second outer member lying in a second plane;
a core extending between the first and second outer members at a location interior to the first and second perimeters, wherein the first and second outer members are maintained with the first and second inner surfaces in opposition about the core with the first and second planes oriented generally in parallel and spaced apart; and
a compressible material disposed on at least a portion of one of the first and second inner surfaces;
wherein the first and second inner surfaces are configured to engage and hold the elongated medical body when the elongated medical body is advanced therebetween towards the core and is in engagement with the compressible material.

2. The holder of claim 1 further comprising:
a writeable surface disposed on the outer surface of one of the first and second outer members.

3. The holder of claim 1 further comprising:
an adhesive disposed on one of the outer surfaces of the first and second outer members
wherein the adhesive is configured to attach to one of the first and second outer surfaces and to a surface not forming part of the holder.

4. The holder of claim 1 wherein the core further comprises a first connector attached to one of the first and second outer members, wherein the first connector is connected to the other of the first and second outer members.

5. The holder of claim 1 wherein the first and second inner surfaces are configured to form a funnel cross sectional shape.

6. A holder for an elongated medical body suitable for insertion into a patient during a medical procedure, comprising:
a first outer member having a first inner surface, and a first outer surface,
a second outer member having a second inner surface and a second outer surface,
a core structure extending between the first and second outer members;
an adhesive member configured to adhere the holder to a first surface;
two holding members respectively placed between the inner surfaces of the first and second outer members, and wherein the holding members are compressible and configured to hold therebetween the elongated medical body; and
wherein the adhesive member is configured to attach to one of the first and second outer surfaces.

7. The holder of claim 6, wherein the core structure comprises a first connection member coupled to the first outer member and a second connection member coupled to the second outer member.

8. The holder of claim 6, further comprising a writable surface on the first and second outer surfaces.

9. The holder of claim 6, wherein each of the first and second outer members have an outer perimeter and a center and a surface that tapers from the outer perimeter to a point between the outer perimeter and the center defining a funnel configured to capture the elongated medical body.

10. The holder of claim 6, wherein the attachment of the first and second outer members defines a space suitable for holding the elongated medical body between the two holding members.

11. The holder of claim 6, wherein two holding members further comprise a foam material.

12. The holder of claim 6, wherein the two holding members further comprise a silicone material.

13. The holder of claim 12, wherein at least one of the holding members further comprise a plurality of protrusions, wherein the protrusions are configured to capture portions of the elongated medical body.

14. The holder of claim 13, wherein at least some of the protrusions are of differing sizes.

15. The holder of claim 6, wherein at least one of the first and second outer members includes a recess on its outer surface relative to its outer edge, wherein the recess comprises a recess surface configured to receive labeling or the adhesive member.

16. The holder of claim 6, wherein the first connection member comprises two prongs and the second connection member comprises two openings, wherein the two prongs are configured to securely fit into and attach to the two openings.

17. The holder of claim 6, wherein the first connection member comprises a protruding member and the second connection member comprises a notch configured to securely receive the protruding member, wherein the first outer member and the second outer member are securely attached to one another having an axis of rotation, and in a locked position upon insertion of the protruding member into the notch and subsequent turning of either the first or the second outer member in a first direction about the axis of rotation.

18. The holder of claim 17, wherein the protruding member comprises a compressible member configured to assist in releasing the first outer member and the second outer member from the locked position upon turning either the first or the second outer member in a second direction about the axis of rotation.

19. The holder of claim 18, wherein the compressible member is a spring.

20. The holder of claim 6, wherein the writable surface comprises a label adhered to the outer surface of the respective outer member.

21. The holder of claim 6, wherein the adhesive member maintains its adhesive qualities upon multiple attachments and detachments from the first surface.

22. The holder of claim 6, wherein the adhesive member is configured to removably attach to the first surface, and wherein the first surface is a drape, a garment, a table, or surgical tray.

23. The holder of claim 6, further comprising a secondary attachment mechanism attached to either the first outer member or the second outer member, wherein the secondary attachment mechanism is configured to attach to a drape or a garment.

24. A method for operating a holder for an elongated medical body, the method comprising:
providing the holder, wherein the holder comprises:
a first outer member having a first inner surface and a first outer surface,
a second outer member having a second inner surface and a second outer surface,
a core structure extending between the first and second outer members;
an adhesive member;
two holding members respectively placed between the inner surfaces of the first and second outer members,
wherein each of the first and second outer members have an outer perimeter and a surface that tapers from the outer perimeter to a point on the respective inner surface defining a funnel configured to capture the elongated medical body;
feeding a portion of the elongated medical body, via the funnel, between the two holding members; and
holding the elongated medical body between the two holding members via friction.

25. The method of claim 24, wherein at least one of the first and second outer members includes a recess on its outer surface relative to its outer edge, wherein the recess is configured to receive a label having a writable surface or the adhesive member.

26. The method of claim 24, further comprising:
attaching the adhesive member to a first surface.

27. The method of claim 26, wherein the adhesive member is configured to removably attach to the first surface, and wherein the first surface is a drape, a garment, a table, or surgical tray.

28. The method of claim 24, wherein the elongated medical body is a guidewire.

29. The method of claim 28, further comprising:
pulling the guidewire to cause a portion of the guidewire held between the two holding members to be released, wherein the pulling of the guidewire causes the portion of the guidewire to be cleaned by the two holding members as it is released.

30. The method of claim 29, further comprising:
applying a cleaning liquid on the holding members.

31. A kit comprising:
the holder of claim 1;
a writing utensil configured to write on a writable surface of the holder, and a removeable covering for the adhesive member of the holder; and
an instrument pad for wiping an elongated medical body.

32. The kit of claim 31, wherein the instrument pad is an antiseptic instrument pad.

33. The kit of claim 31, further comprising at least one elongated medical body, wherein the at least one elongated medical body is a guidewire or a catheter.

34. The kit of claim 31, further comprising a pouch card configured for selective attachment to the holder, wherein the pouch card is removably fastened to an attachment mechanism.
